(19) United States
(12) Patent Application Publication
(10) Pub. No.: US 2020/0165685 A1
(43) Pub. Date: May 28, 2020

(54) CIRCULATING RNA FOR DETECTION, PREDICTION, AND MONITORING OF CANCER

(71) Applicant: NANTOMICS, LLC, Culver City, CA (US)

(72) Inventors: Shahrooz RABIZADEH, Culver City, CA (US); Patrick SOON-SHIONG, Culver City, CA (US); Kathleen DANENBERG, Culver City, CA (US)

(21) Appl. No.: 16/611,834

(22) PCT Filed: May 9, 2018
(86) PCT No.: PCT/US2018/031764

§ 371 Date: Nov. 7, 2019

Related U.S. Application Data
(60) Provisional application No. 62/504,149, filed on May 10, 2017.
(60) Provisional application No. 62/511,849, filed on May 26, 2017.
(60) Provisional application No. 62/513,706, filed on Jun. 1, 2017.
(60) Provisional application No. 62/582,862, filed on Nov. 7, 2017.

(51) Int. Cl. C12Q 1/6886; C12Q 1/686

(52) U.S. Cl. CPC C12Q 1/6886; C12Q 1/686; C12Q 2600/156; C12Q 2561/113; C12Q 2600/112; C12Q 2600/106; C12Q 2600/158

(57) ABSTRACT

Circulating free RNA (cfRNA) and/or circulating tumor RNA (ctRNA) are employed to identify and quantitate expression levels of various genes and further allows for non-invasive monitoring of changes in such genes. Moreover, analysis of ct/cfRNA (and ct/cfDNA) enable detection, prediction, and monitoring of cancer status based on the presence of circulating free cfRNA and/or ctRNA, and further identify or determine a treatment and the response to the treatment.

This application claims priority to our co-pending US provisional applications having the Ser. No. 62/504,149, filed May 10, 2017, the Ser. No. 62/511,849, filed May 26, 2017, the Ser. No. 62/513,706, filed Jun. 1, 2017, and the Ser. No. 62/582,862, filed Nov. 7, 2017, which are incorporated in their entireties herein.

### FIELD OF THE INVENTION

The field of the invention is systems and methods of determining cancer status by detecting and/or quantifying circulating tumor RNA and/or circulating cell free RNA of cancer-related genes.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Efforts in improving cancer treatment have largely focused on screening, development of new anti-cancer agents, multi-drug combinations, and advances in radiation therapy. A more recent approach is precision medicine, which takes individual variability into account to design personalized treatment strategies. One important goal of precision medicine is to identify molecular markers indicative of therapy selection by analyzing the factors involved in the therapeutic effects and prognosis. So far, such information has been obtained by analysis of genes and proteins from cancer tissue biopsies.

However, the use of tissue biopsies has many problems, including possible sampling bias and a limited ability to monitor tumor markers in patients during the course of the therapy. In 1977, Leon et al. discovered that serum circulating tumor DNA (ctDNA) levels were higher in some patients with cancer, suggesting that the extra serum DNA in cancer patients originates from their tumor. Subsequent work confirmed this hypothesis and established that ctDNA could in at least some cases reveal the same information about the patient's genes as that found in the tumor without an invasive tissue biopsy. Further studies revealed that the genetic information from liquid biopsies could originate from various sources, including circulating cancer cells (CTC) and exosomes.

While many studies have described the use of ctDNA to study cancer genomes and monitoring or diagnosing cancer, relatively few studies have used ctRNA. Advantageously, the ctRNA may at least potentially contain the same mutational information as ctDNA, but is present only for genes that are actually expressed. In addition, ctRNA could also at least conceptually provide information about the quantitative expression levels of genes (i.e., the amount of transcription into mRNA). However, RNA is known to be highly unstable, and at least for this reason was not subject to much investigation. Therefore, most of the work associated with RNA was focused on biopsy materials and associated protocols to detect and/or quantify RNA in such materials, including RNAseq, RNA hybridization panels, etc. Unfortunately, biopsies are often not readily available and subject the patient to added risk.

To circumvent such difficulties, selected cfRNA tests have focused on detecting already known markers specific to certain tumors. For example, U.S. Pat. No. 9,469,876 to Kuslich and U.S. Pat. No. 8,597,892 to Shelton discuss detecting circulating microRNA biomarkers associated with circulating vesicles in the blood for diagnosis of a specific type of cancer (e.g., prostate cancer, etc.). In another example, U.S. Pat. No. 8,440,396 to Kopreski discloses detection of circulating mRNA fragment of genes encoding tumor associated antigens that are known as markers of several types of cancers (e.g., melanoma, leukemia, etc.). Yet, such approaches are often limited to provide piecemeal information on the prognosis of the cancer such that, for example, the status and many cancer conditions that are indirectly associated with or caused by the cancer cell (e.g., presence of metastasis, presence of cancer stem cells, presence of immune suppressive tumor microenvironment, increased or decreased activity of an immune competent cell against the cancer, etc.) cannot be associated.

Therefore, even though numerous methods of nucleic acid analysis from biological fluids are known in the art, all or almost all of them suffer from various disadvantages. Consequently, there remains a need for improved systems and methods to isolate circulating nucleic acids, and especially ctRNA to determine the status and other conditions that are indirectly associated with or caused by the cancer cell.

### SUMMARY OF THE INVENTION

The inventive subject matter is directed to systems and methods related to blood-based RNA expression testing that identifies, and/or quantitates expression, and that allows for non-invasive monitoring of changes in drivers of disease or conditions of the microenvironment of or around the diseased tissue that have heretofore only been available by protein-based analysis of biopsied tissue. Advantageously, such methods allow for identification or prognosis of status and other cancer conditions that are indirectly associated with or caused by the cancer cell.

Preferred RNA expression testing is performed via detection and/or quantification of circulating tumor RNA (ctRNA) and/or circulating free RNA (cfRNA), which may be informed by (and in some cases replaced by) detection and/or quantification of circulating tumor DNA (ctDNA) and/or circulating free DNA (cfDNA). The RNA expression will typically be based on or include disease related genes, wherein these genes may be in wild type, mutated (e.g., patient specific mutation, including SNPs, neoepitopes, fusions, etc.) and/or splice variant forms.

Thus, it should be appreciated that contemplated systems and methods advantageously allow detection of onset and/or progression of disease, detection and analysis of tumor microenvironment condition, detection and analysis of molecular changes of the tumor cells, identification of changes in drug targets that may be associated with emerging resistance to various treatment modalities, or prediction of likely treatment outcome using various treatment modalities. Moreover, contemplated systems and methods advantageously integrate with other omics analysis platforms, and especially GPS Cancer, to establish a powerful primary analysis/monitoring combination tool in which alterations identified by an omics platform are non-invasively, molecularly monitored by systems and methods presented herein.

In one aspect of the inventive subject matter, the inventors contemplate method of determining cancer status in an individual having or suspected to have a cancer. In this method, a sample of a bodily fluid of the individual is obtained and a quantity of at least one of cfRNA and ctRNA in the sample is determined. Most preferably, the cfRNA and ctRNA is derived from a cancer related gene. Then, the quantity of the at least one of cfRNA and ctRNA is associated with the cancer status.

In preferred aspects, the cancer related gene is one or more of ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCB5, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGR5, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, XCR1, and DCC, UNC5A, Netrin, and IL8. Of course, it should be appreciated that the above genes may be wild type or mutated versions, including missense or nonsense mutations, insertions, deletions, fusions, and/or translocations, all of which may or may not cause formation of a neoepitope in a protein expressed from such RNA.

With respect to the cancer status it is contemplated that suitable status include types of cancer (e.g., solid cancer), anatomical location of the cancer, clonality evolution of cancer cell, susceptibility of the cancer to treatment with a drug, presence or absence of the cancer in the individual, presence of metastasis, presence of cancer stem cells, presence of immune suppressive tumor microenvironment, and increased or decreased activity of an immune competent cell against the cancer. Moreover, it is generally contemplated that the cancer related gene is a cancer associated gene, a cancer specific gene, a cancer driver gene, or a gene encoding a patient and tumor specific neoepitope. For example, the cancer-related gene encodes is a checkpoint inhibition related gene, an epithelial to mesenchymal transition-related gene, an immune suppression-related gene

In some embodiments, suitable cancer related genes may have a patient-specific mutation or may have a patient- and tumor-specific mutation, and the ctRNA or cfRNA can be a portion of the transcript of the cancer related gene encoding the patient-specific and cancer-specific neoepitope. Among other changes, contemplated mutations include missense mutations, insertions, deletions, translocations, fusions, all of which may create a neoepitope in a protein encoded by the cfRNA or ctRNA.

Most typically, the step of quantifying will include isolation of the cfRNA and/or ctRNA (e.g., from blood, serum, plasma, or urine) under conditions and using RNA stabilization agents that substantially avoids cell lysis. Additionally, it is contemplated that the step of quantifying will include real time quantitative PCR of a cDNA prepared from the cfRNA and/or ctRNA. In further preferred methods, the step of associating includes a step of designating the cancer as treatable with a drug or designating the cancer as treatment resistant.

As needed, it is further contemplated that the methods presented herein may also include a step of determining a total quantity of all or substantially all cfRNA and ctRNA in the sample, and optionally a step of associating the determined total quantity with presence or absence of cancer. Additionally, it is also contemplated that the method may further include a step of determining at least one of presence and quantity of a tumor-associated peptide in the sample (e.g., soluble NKG2D).

Optionally, the method may also include determining quantities of at least two of cfRNA and ctRNA in the sample where at least two of cfRNA and ctRNA are derived from two distinct cancer related genes. In such method, a ratio between the quantities of the at least two of cfRNA and ctRNA can be determined and the determined ratio can be associated with the cancer status. In some embodiments, the at least two of cfRNA and ctRNA comprises at least one cfRNA and at least one ctRNA in the sample, and the at least one cfRNA is derived from an immune cell (e.g., suppressive immune cell, etc.).

Still further, the method may also include a step of determining nucleic acid sequence of the at least one of cfRNA and ctRNA. In this method, at least one of cfDNA and ctDNA, which are derived from the same gene with the at least one of cfRNA and ctRNA. In some embodiments, a mutation in a nucleic acid sequence of the at least one of cfDNA and ctDNA can be determined and the mutation and the quantity of at least one of cfRNA and ctRNA can be associated with the cancer status.

Additionally, the method also may include a step of selecting a treatment regimen based on the cancer status. In this method, the treatment regimen comprises a treatment targeting a portion of a peptide encoded by the cancer related gene when the quantity of the at least one of cfRNA and ctRNA derived from the cancer related gene increases. If the at least one of cfRNA and ctRNA is a miRNA, it is contemplated that the treatment regime is an inhibitor to the miRNA.

In yet another aspect of the inventive subject matter, the inventors contemplate a method of treating a cancer. IN this method, at least one of respective cfRNA and ctRNA of first and second marker genes in a blood sample of a patient is determined. Preferably, the first marker gene is a cancer related gene, and the second marker gene is a checkpoint inhibition related gene. Then, using the quantity of the cfRNA or ctRNA derived from the first or second marker gene, a treatment with a first or second pharmaceutical composition, respectively is determined. Preferably, the second pharmaceutical composition comprises a checkpoint inhibitor. Most typically, the cancer related gene is selected form the group consisting of ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, XCR1, DCC, UNC5A, Netrin, CXCR1, CXCR2, and IL8.

For example, the second marker gene may be those encoding PD-1 or PD-L1 and the first pharmaceutical composition may be an immune therapeutic composition or a chemotherapeutic composition. Contemplated methods may further include a step of determining a total quantity of all of at least one of cfRNA and ctRNA in the patient blood sample. Preferably, the step of determining will include a step of isolating the at least one of cfRNA and ctRNA under conditions and using RNA stabilization agents that substantially avoids cell lysis. As noted above, contemplated methods may also include a step of quantifying at least one of cfDNA and ctDNA of a cancer related gene in the blood sample of the patient.

Still another aspect of the inventive subject matter includes a method of generating or updating a patient record of an individual having or suspected to have a cancer. In this method, a sample of a bodily fluid of the individual is obtained, and a quantity of at least one of cfRNA and ctRNA in the sample is determined. Preferably the at least one of cfRNA and ctRNA is derived from a cancer related gene. Then, the quantity of the at least one of cfRNA and ctRNA is associated with the cancer status. The patient record can be generated or updated based on the cancer status. Most typically, the cancer related gene is selected form the group consisting of ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C100ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, XCR1, DCC, UNC5A, Netrin, CXCR1, CXCR2, and IL8.

In still another aspect of the inventive subject matter, the inventors contemplate a method of determining a likelihood of success of an immune therapy to an individual having a cancer. IN this method, a sample of a bodily fluid of the individual is obtained and a quantity of at least one of cfRNA and ctRNA in the sample is determined. Preferably, the cfRNA and ctRNA is derived from at least one of an epithelial to mesenchymal transition-related gene and an immune suppression-related gene. Then the quantity of the at least one of cfRNA and ctRNA is associated with a tumor microenvironment status. The likelihood of success of the immune therapy or treatability of the cancer with the immune therapy can be determined based on a type of the immune therapy and the tumor microenvironment status.

Typically, the tumor microenvironment status is at least one of presence of cancer stem cells, presence of immune suppressive tumor microenvironment, and increased or decreased activity of an immune competent cell against the cancer. Thus, the type of the immune therapy may include a neoepitope-based immune therapy, a checkpoint inhibitor, a regulatory T cell inhibitor, a binding molecule to a cytokine or chemokine, and a cytokine or chemokine, a miRNA inhibiting epithelial to mesenchymal transition. In some embodiment, the immune therapy is determined to have a high likelihood of success where the quantity of the at least one of cfRNA and ctRNA is below a predetermined threshold. Additionally, the method may also include a step of administering the immune therapy to the individual where the quantity of the at least one of cfRNA and ctRNA is below a predetermined threshold.

Various objects, features, aspects and advantages of the inventive subject matter will become more apparent from the following detailed description of preferred embodiments and accompanied drawings.

### BRIEF DESCRIPTION OF THE DRAWING

FIG. 1 depicts graphs comparing plasma concentrations for cfDNA and cfRNA for healthy subjects and subjects diagnosed with cancer.

FIG. 2 depicts a graph of ctRNA expression levels in the plasma of patients progressing on various therapies.

FIG. 3 depicts a graph showing PD-L1 cfRNA levels for a non-responder and a responder to nivolumab and corresponding IHC staining of lung tumor samples, along with PD-L1 cfRNA levels during treatment.

FIG. 4 provides a schematic showing of presence of PD-L1 ctRNA upon Nivolumab treatment in a patient.

FIG. 5 depicts a graph correlating PD-L1 cfRNA levels with the PD-L1 status as determined by PD-L1 IHC

FIG. 6 depicts graphs comparing PD-L1 cfRNA expression in two patients treated with Nivolumab.

FIG. 7 depicts a graph showing the relative expression of PD-L1 cfRNA for lung cancer patients in a clinical trial and a table summarizing the data.

FIG. 8A depicts a graph comparing plasma concentrations for PD-L1 cfRNA for across various cancer types or with a healthy individual, respectively.

FIG. 8B depicts a graph showing plasma concentrations for PD-L1 cfRNA for healthy subjects.

FIG. 9A depicts a graph showing relative co-expression of PD-L1 and HER2 in gastric cancer as measured by cfRNA levels.

FIG. 9B depicts a graph showing relative co-expression of PD-L1 and HER2 as measured by cfRNA levels.

FIG. 10 depicts a schematic diagram of Androgen receptor splice variant 7 (AR-V7).

FIG. 11 depicts exemplary results for AR-V7 cfRNA levels and AR cfRNA levels in prostate cancer patients indicating that AR-V7 cfRNA is a suitable marker.

FIG. 12 depicts a graph showing relative coexpression of LAC-3, PD-L1, TIM-3 as measured by cfRNA levels in multiple prostate cancer patients.

FIG. 13 depicts a graph showing PCA3 cfRNA expression in prostate cancer patients compared to non-prostate cancer patient.

### DETAILED DESCRIPTION

The inventors contemplate that tumor cells and/or some immune cells interacting or surrounding the tumor cells release cfRNA, more specifically ctRNA to the patient's bodily fluid, and thus may increase the quantity of the specific ctRNA in the patient's bodily fluid as compared to a healthy individual. Given that, the inventors have now discovered that ctRNA and/or cfRNA can be employed as a sensitive, selective, and quantitative marker for diagnosis, indication and/or a change in specific tumor microenvironment or cell status, monitoring of treatment, identifying or recommending a treatment with high likelihood of success, and even as discovery tool that allows repeated and non-invasive sampling of a patient. In this context, it should be noted that the total cfRNA will include ctRNA, wherein the ctRNA may have a patient and tumor specific mutation and as such be distinguishable from the corresponding cfRNA of healthy cells, or wherein the ctRNA may be selectively expressed in tumor cells and not be expressed in corresponding healthy cells.

Viewed from a different perspective, the inventors therefore discovered that various nucleic acids, more specifically cfDNA/cfRNAs, or further specifically ctDNA/ctRNAs, may be selected for detection and/or monitoring a status of a tumor, more specifically a molecular or cellular status of tumor cell and/or tumor microenvironment, prognosis of tumor, recommendation of suitable treatment and treatment plan, and treatment response/effectiveness of a treatment regimen in a particular patient.

Consequently, in one especially preferred aspect of the inventive subject matter, the inventors contemplate a method of determining or monitoring a cancer status in an individual having or suspected to have a cancer. In this method, a sample of a bodily fluid of the individual is obtained and, from the sample of the bodily fluid, a quantity of at least one of cfRNA and ctRNA is determined.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

Most typically, suitable bodily fluid to obtain cfDNA/cfRNAs includes whole blood, which is preferably provided as plasma or serum. Thus, in a preferred embodiment, the cfDNA/cfRNAs is isolated from a whole blood sample that is processed under conditions that preserve cellular integrity and stability of cfDNA/cfRNAs. Alternatively, it should be noted that various other bodily fluids are also deemed appropriate so long as ctRNA and/or cfRNA is present in such fluids. Appropriate fluids include saliva, ascites fluid, spinal fluid, urine, or any other types of bodily fluid, which may be fresh, chemically preserved, refrigerated or frozen.

The bodily fluid of the patient can be obtained at any desired time point(s) depending on the purpose of the omics analysis. For example, the bodily fluid of the patient can be obtained before and/or after the patient is confirmed to have a tumor and/or periodically thereafter (e.g., every week, every month, etc.) in order to associate the ctDNA and/or ctRNA data with the prognosis of the cancer. In some embodiments, the bodily fluid of the patient can be obtained from a patient before and after the cancer treatment (e.g., chemotherapy, radiotherapy, drug treatment, cancer immunotherapy, etc.). While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.).

Additionally or alternatively, the bodily fluid of a healthy individual can be obtained to compare the sequence/modification of cfDNA and/or cfRNA sequence, and/or quantity/subtype expression of the cfRNA. As used herein, a healthy individual refers an individual without a tumor. Preferably, the healthy individual can be chosen among group of people shares characteristics with the patient (e.g., age, gender, ethnicity, diet, living environment, family history, etc.).

Any suitable methods for isolating cell free DNA/RNA are contemplated. For example, in one exemplary method of DNA isolation, specimens were accepted as 10 ml of whole blood drawn into a test tube. Cell free DNA can be isolated from other from mono-nucleosomal and di-nucleosomal complexes using magnetic beads that can separate out cell free DNA at a size between 100-300 bps. For another example, in one exemplary method of RNA isolation, specimens were accepted as 10 ml of whole blood drawn into cell-free RNA BCT® tubes or cell-free DNA BCT® tubes containing RNA stabilizers, respectively. Advantageously, cell free RNA is stable in whole blood in the cell-free RNA BCT tubes for seven days while cell free RNA is stable in whole blood in the cell-free DNA BCT Tubes for fourteen days, allowing time for shipping of patient samples from world-wide locations without the degradation of cell free RNA.

It is generally preferred that the cfRNA is isolated using RNA stabilization reagents. While any suitable RNA stabilization agents are contemplated, preferred RNA stabilization reagents include one or more of a nuclease inhibitor, a preservative agent, a metabolic inhibitor, and/or a chelator. For example, contemplated nuclease inhibitors may include RNAase inhibitors such as diethyl pyrocarbonate, ethanol, aurintricarboxylic acid (ATA), formamide, vanadyl-ribonucleoside complexes, macaloid, heparin, bentonite, ammonium sulfate, dithiothreitol (DTT), beta-mercaptoethanol, dithioerythritol, tris(2-carboxyethyl)phosphene hydrochloride, most typically in an amount of between 0.5 to 2.5 wt %. Preservative agents may include diazolidinyl urea (DU), imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-1 aza-3,7-dioxabicyclo[3.3.0]octane, 5-hydroxymethyl-1-1aza-3,7dioxabicyclo[3.3.0]octane, 5-hydroxypoly[methyleneoxy]methyl-1-1-aza-3,7-dioxabicyclo [3.3.0]octane, quaternary adamantine or any combination thereof. In most examples, the preservative agent will be present in an amount of about 5-30 wt %. Moreover, it is generally contemplated that the preservative agents are free of chaotropic agents and/or detergents to reduce or avoid lysis of cells in contact with the preservative agents.

Suitable metabolic inhibitors may include glyceraldehyde, dihydroxyacetone phosphate, glyceraldehyde 3-phosphate, 1,3-bisphosphoglycerate, 3-phosphoglycerate, phosphoenolpyruvate, pyruvate, and glycerate dihydroxyacetate, and sodium fluoride, which concentration is typically in the range of between 0.1-10 wt %. Preferred chelators may include chelators of divalent cations, for example, ethylenediaminetetraacetic acid (EDTA) and/or ethylene glycol-bis(β-aminoethyl ether)-N,N,N′,N′-tetraacetic acid (EGTA), which concentration is typically in the range of between 1-15 wt %.

Additionally, RNA stabilizing reagent may further include protease inhibitors, phosphatase inhibitors and/or polyamines. Therefore, exemplary compositions for collecting and stabilizing ctRNA in whole blood may include aurintricarboxylic acid, diazolidinyl urea, glyceraldehyde/sodium fluoride, and/or EDTA. Further compositions and methods for ctRNA isolation are described in U.S. Pat. Nos. 8,304,187 and 8,586,306, which are incorporated by reference herein.

Most preferably, such contemplated RNA stabilization agents for ctRNA stabilization are disposed within a test tube that is suitable for blood collection, storage, transport, and/or centrifugation. Therefore, in most typical aspects, the collection tube is configured as an evacuated blood collection tube that also includes one or more serum separator substance to assist in separation of whole blood into a cell containing and a substantially cell free phase (no more than 1% of all cells present). In general, it is preferred that the RNA stabilization agents do not or substantially do not (e.g., equal or less than 1%, or equal or less than 0.1%, or equal or less than 0.01%, or equal or less than 0.001%, etc.) lyse blood cells. Viewed from a different perspective, RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In some aspects, contemplated reagents will stabilize ctRNA in a collected tissue other than blood for at 2 days, more preferably at least 5 days, and most preferably at least 7 days. Of course, it should be recognized that numerous other collection modalities other than collection tube (e.g., a test plate, a chip, a collection paper, a cartridge, etc.) are also deemed appropriate, and that the ctDNA and/or ctRNA can be at least partially purified or adsorbed to a solid phase to so increase stability prior to further processing.

As will be readily appreciated, fractionation of plasma and extraction of cfDNA and/or cfRNA can be done in numerous manners. In one exemplary preferred aspect, whole blood in 10 mL tubes is centrifuged to fractionate plasma at 1600 rcf for 20 minutes. The so obtained clarified plasma fraction is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). ctDNA and ctRNA are extracted from 2 mL of plasma using commercially available Qiagen reagents. For example, where cfRNA was isolated, the inventors used a second container that included a DNase that was retained in a filter material. Notably, the cfRNA also included miRNA (and other regulatory RNA such as shRNA, siRNA, and intronic RNA). Therefore, it should be appreciated that contemplated compositions and methods are also suitable for analysis of miRNA and other RNAs from whole blood.

Moreover, it should also be recognized that the extraction protocol was designed to remove potential contaminating blood cells, other impurities, and maintain stability of the nucleic acids during the extraction. All nucleic acids were kept in bar-coded matrix storage tubes, with ctDNA stored at −4° C. and ctRNA stored at −80° C. or reverse-transcribed to cDNA (e.g., using commercially reverse transcriptase such as Maxima or Superscript VILO) that is then stored at −4° C. or refrigerated at +2-8° C. Notably, so isolated ctRNA can be frozen prior to further processing.

It is contemplated that cfDNA and cfRNA may include any types of DNA/RNA that are originated or derived from tumor cells that are circulating in the bodily fluid of a person without being enclosed in a cell body or a nucleus. While not wishing to be bound by a particular theory, it is contemplated that release of cfDNA/cfRNA can be increased when the tumor cell interacts with an immune cell or when the tumor cells undergo cell death (e.g., necrosis, apoptosis, autophagy, etc.). Thus, in some embodiments, cfDNA/cfRNA may be enclosed in a vesicular structure (e.g., via exosomal release of cytoplasmic substances) so that it can be protected from nuclease (e.g., RNase) activity in some type of bodily fluid. Yet, it is also contemplated that in other aspects, the cfDNA/cfRNA is a naked DNA/RNA without being enclosed in any membranous structure, but may be in a stable form by itself or be stabilized via interaction with one or more non-nucleotide molecules (e.g., any RNA binding proteins, etc.).

Thus, the cfDNA may include any whole or fragmented genomic DNA, or mitochondrial DNA, and the cfRNA may include mRNA, tRNA, microRNA, small interfering RNA, long non-coding RNA (1ncRNA). Most typically, the cell free DNA is a fragmented DNA typically with a length of at least 50 base pair (bp), 100 bp, 200 bp, 500 bp, or 1 kbp. Also, it is contemplated that the cfRNA is a full length or a fragment of mRNA (e.g., at least 70% of full-length, at least 50% of full length, at least 30% of full length, etc. In some embodiments, the ctDNA and ctRNA are fragments that may correspond to the same or substantially similar portion of the gene (e.g., at least 50%, at least 70%, at least 90% of the ctRNA sequence is complementary to ctDNA sequence, etc.). In other embodiments, the ctDNA and ctRNA are fragments may correspond to different portion of the gene (e.g., less than 50%, less than 30%, less than 20% of the ctRNA sequence is complementary to ctDNA sequence, etc.). While less preferred, it is also contemplated that the ctDNA and cell free RNA may be derived from different genes from the tumor cell. In some embodiments, it is also contemplated that the ctDNA and cfRNA may be derived from different genes from the different types of cells (e.g., ctDNA from the tumor cell and cfRNA from the NK cell, etc.).

While cfDNA/cfRNA may include any type of DNA/RNA encoding any cellular, extracellular proteins or non-protein elements, it is preferred that at least some of cfDNA/cfRNA encodes one or more cancer-related proteins, inflammation-related proteins, DNA repair-related proteins, or RNA repair-related proteins, which mutation, expression and/or function may directly or indirectly be associated with tumorigenesis, metastasis, formation of immune suppressive tumor microenvironment, immune evasion, epithelial-mesenchymal transition, or presentation of patient-, tumor-specific neoepitope on the tumor cell. It is also contemplated that the cfDNA/cfRNA may be derived from one or more genes encoding cell machinery or structural proteins including, but not limited to, housekeeping genes, transcription factors, repressors, RNA splicing machinery or elements, translation factors, tRNA synthetases, RNA binding protein, ribosomal proteins, mitochondrial ribosomal proteins, RNA polymerase, proteins related to protein processing, heat shock proteins, cell cycle-related proteins, elements related to carbohydrate metabolism, lipid, citric acid cycle, amino acid metabolism, NADH dehydrogenase, cytochrome c oxidase, ATPase, lysosome, proteasome, cytoskeletal proteins and organelle synthesis. Thus, for example, cfDNA/cfRNA can be derived from genes, including, but not limited to, ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCB5, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGR5, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, XCR1, DCC, UNC5A, Netrin, and IL-8.

In another example, cfDNA/cfRNA can be derived from genes encoding one or more inflammation-related proteins, including, but not limited to, HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, and hTERT, and in yet another example, the ctRNA encoded a full length or a fragment of HMGB1.

In still another example, cfDNA/cfRNA can be derived from genes encoding DNA repair-related proteins or RNA repair-related proteins. Table 1 provides an exemplary collection of predominant RNA repair genes and their associated repair pathways contemplated herein, but it should be recognized that numerous other genes associated with DNA repair and repair pathways are also expressly contemplated herein, and Tables 2 and 3 illustrate further exemplary genes for analysis and their associated function in DNA repair.

TABLE 1

| Repair mechanism | Predominant DNA Repair genes |
|---|---|
| Base excision repair (BER) | DNA glycosylase, APE1, XRCC1, PNKP, Tdp1, APTX, DNA |
| | polymerase β, FEN1, DNA polymerase δ or ε, PCNA-RFC, |
| | PARP |
| Mismatch repair (MMR) | MutSα (MSH2-MSH6), MutSβ (MSH2-MSH3), MutLα |
| | (MLH1-PMS2), MutLβ (MLH1-PMS2), MutLγ (MLH1- |
| | MLH3), Exo1, PCNA-RFC |
| Nucleotide excision repair | XPC-Rad23B-CEN2, UV-DDB (DDB1-XPE), CSA, CSB, |
| (NER) | TFIIH, XPB, XPD, XPA, RPA, XPG, ERCC1-XPF, DNA |
| | polymerase δ or ε |
| Homologous recombination | Mre11-Rad50-Nbs1, CtIP, RPA, Rad51, Rad52, BRCA1, |
| (HR) | BRCA2, Exo1, BLM-TopIIIα, GEN1-Yen1, Slx1-Slx4, |
| | Mus81/Eme1 |
| Non-homologous end-joining | Ku70-Ku80, DNA-PKc, XRCC4-DNA ligase IV, XLF |
| (NHEJ) | |

TABLE 2

| | | Accession |
|---|---|---|
| Gene name (synonyms) | Activity | number |
| Base excision repair | | |
| (BER) | | |
| | DNA glycosylases: major altered | |
| | base released | |
| UNG | U excision | NM_003362 |
| SMUG1 | U excision | NM_014311 |
| MBD4 | U or T opposite G at CpG | NM_003925 |
| | sequences | |
| TDG | U, T or ethenoC opposite G | NM_003211 |
| OGG1 | 8-oxoG opposite C | NM_002542 |
| MYH | A opposite 8-oxoG | NM_012222 |
| NTH1 | Ring-saturated or fragmented | NM_002528 |
| | pyrimidines | |
| MPG | 3-meA, ethenoA, hypoxanthine | NM_002434 |
| | Other BER factors | |
| APE1 (HAP1, APEX, | AP endonuclease | NM_001641 |
| REF1) | | |
| APE2 (APEXL2) | AP endonuclease | NM_014481 |
| LIG3 | Main ligation function | NM_013975 |
| XRCC1 | Main ligation function | NM_006297 |
| | Poly(ADP-ribose) polymerase | |
| | (PARP) enzymes | |
| ADPRT | Protects strand interruptions | NM_001618 |
| ADPRTL2 | PARP-like enzyme | NM_005485 |
| ADPRTL3 | PARP-like enzyme | AF085734 |
| Direct reversal of damage | | |
| MGMT | O6-meG alkyltransferase | NM_002412 |
| Mismatch excision repair | | |
| (MMR) | | |
| MSH2 | Mismatch and loop recognition | NM_000251 |
| MSH3 | Mismatch and loop recognition | NM_002439 |
| MSH6 | Mismatch recognition | NM_000179 |
| MSH4 | MutS homolog specialized for | NM_002440 |
| | meiosis | |
| MSH5 | MutS homolog specialized for | NM_002441 |
| | meiosis | |
| PMS1 | Mitochondrial MutL homolog | NM_000534 |
| MLH1 | MutL homolog | NM_000249 |
| PMS2 | MutL homolog | NM_000535 |
| MLH3 | MutL homolog of unknown | NM_014381 |
| | function | |
| PMS2L3 | MutL homolog of unknown | D38437 |
| | function | |
| PMS2L4 | MutL homolog of unknown | D38438 |
| | function | |
| Nucleotide excision repair | | |
| (NER) | | |
| XPC | Binds damaged DNA as complex | NM_004628 |
| RAD23B (HR23B) | Binds damaged DNA as complex | NM_002874 |
| CETN2 | Binds damaged DNA as complex | NM_004344 |
| RAD23A (HR23A) | Substitutes for HR23B | NM_005053 |
| XPA | Binds damaged DNA in preincision | NM_000380 |
| | complex | |
| RPA1 | Binds DNA in preincision complex | NM_ 002945 |
| RPA2 | Binds DNA in preincision complex | NM_002946 |
| RPA3 | Binds DNA in preincision complex | NM_002947 |
| TFIIH | Catalyzes unwinding in preincision | |
| | complex | |
| XPB (ERCC3) | 3′ to 5′ DNA helicase | NM_000122 |
| XPD (ERCC2) | 5′ to 3′ DNA helicase | X52221 |
| GTF2H1 | Core TFIIH subunit p62 | NM_005316 |
| GTF2H2 | Core TFIIH subunit p44 | NM_001515 |
| GTF2H3 | Core TFIIH subunit p34 | NM_001516 |
| GTF2H4 | Core TFIIH subunit p52 | NM_001517 |
| CDK7 | Kinase subunit of TFIIH | NM_001799 |
| CCNH | Kinase subunit of TFIIH | NM_001239 |
| MNAT1 | Kinase subunit of TFIIH | NM_002431 |
| XPG (ERCC5) | 3′ incision | NM_000123 |
| ERCC1 | 5′ incision subunit | NM_001983 |
| XPF (ERCC4) | 5′ incision subunit | NM_005236 |
| LIG1 | DNA joining | NM_000234 |
| NER-related | | |
| CSA (CKN1) | Cockayne syndrome; needed for | NM_000082 |
| | transcription-coupled NER | |
| CSB (ERCC6) | Cockayne syndrome; needed for | NM_000124 |
| | transcription-coupled NER | |
| XAB2 (HCNP) | Cockayne syndrome; needed for | NM_020196 |
| | transcription-coupled NER | |
| DDB1 | Complex defective in XP group E | NM_001923 |
| DDB2 | Mutated in XP group E | NM_000107 |
| MMS19 | Transcription and NER | AW852889 |
| Homologous | | |
| recombination | | |
| RAD51 | Homologous pairing | NM_002875 |
| RAD51L1 (RAD51B) | Rad51 homolog | U84138 |
| RAD51C | Rad51 homolog | NM_002876 |
| RAD51L3 (RAD51D) | Rad51 homolog | NM_002878 |
| DMC1 | Rad51 homolog, meiosis | NM_007068 |
| XRCC2 | DNA break and cross-link repair | NM_005431 |
| XRCC3 | DNA break and cross-link repair | NM_005432 |
| RAD52 | Accessory factor for recombination | NM_002879 |
| RAD54L | Accessory factor for recombination | NM_003579 |
| RAD54B | Accessory factor for recombination | NM_012415 |
| BRCA1 | Accessory factor for transcription | NM_007295 |
| | and recombination | |
| BRCA2 | Cooperation with RAD51, essential | NM_000059 |
| | function | |
| RAD50 | ATPase in complex with MRE11A, | NM_005732 |
| | NBS1 | |
| MRE11A | 3′ exonuclease | NM_005590 |
| NBS1 | Mutated in Nijmegen breakage | NM_002485 |
| | syndrome | |
| Nonhomologous end- | | |
| joining | | |
| Ku70 (G22P1) | DNA end binding | NM_001469 |
| Ku80 (XRCC5) | DNA end binding | M30938 |
| PRKDC | DNA-dependent protein kinase | NM_006904 |
| | catalytic subunit | |
| LIG4 | Nonhomologous end-joining | NM_002312 |
| XRCC4 | Nonhomologous end-joining | NM_003401 |
| Sanitization of nucleotide | | |
| pools | | |
| MTH1 (NUDT1) | 8-oxoGTPase | NM_002452 |
| DUT | dUTPase | NM_001948 |
| DNA polymerases | | |
| (catalytic subunits) | | |
| POLB | BER in nuclear DNA | NM_002690 |
| POLG | BER in mitochondrial DNA | NM_002693 |
| POLD1 | NER and MMR | NM_002691 |
| POLE1 | NER and MMR | NM_006231 |
| PCNA | Sliding clamp for pol delta and pol | NM_002592 |
| | epsilon | |
| REV3L (POLZ) | DNA pol zeta catalytic subunit, | NM_002912 |
| | essential function | |
| REV7 (MAD2L2) | DNA pol zeta subunit | NM_006341 |
| REV1 | dCMP transferase | NM_016316 |
| POLH | XP variant | NM_006502 |
| POLI (RAD30B) | Lesion bypass | NM_007195 |
| POLQ | DNA cross-link repair | NM_006596 |
| DINB1 (POLK) | Lesion bypass | NM_016218 |
| POLL | Meiotic function | NM_013274 |
| POLM | Presumed specialized lymphoid | NM_013284 |
| | function | |
| TRF4-1 | Sister-chromatid cohesion | AF089896 |
| TRF4-2 | Sister-chromatid cohesion | AF089897 |
| Editing and processing | | |
| nucleases | | |
| FEN1 (DNase IV) | 5′ nuclease | NM_004111 |
| TREX1 (DNase III) | 3′ exonuclease | NM_007248 |
| TREX2 | 3′ exonuclease | NM_007205 |
| EX01 (HEX1) | 5′ exonuclease | NM_003686 |
| SPO11 | endonuclease | NM_012444 |
| Rad6 pathway | | |
| UBE2A (RAD6A) | Ubiquitin-conjugating enzyme | NM_003336 |
| UBE2B (RAD6B) | Ubiquitin-conjugating enzyme | NM_003337 |
| RAD18 | Assists repair or replication of | AB035274 |
| | damaged DNA | |
| UBE2VE (MMS2) | Ubiquitin-conjugating complex | AF049140 |
| UBE2N (UBC13, BTG1) | Ubiquitin-conjugating complex | NM_003348 |
| Genes defective in | | |
| diseases associated with | | |
| sensitivity to DNA | | |
| damaging agents | | |
| BLM | Bloom syndrome helicase | NM_000057 |
| WRN | Werner syndrome helicase/3′- | NM_000553 |
| | exonuclease | |
| RECQL4 | Rothmund-Thompson syndrome | NM_004260 |
| ATM | Ataxia telangiectasia | NM_000051 |
| Fanconi anemia | | |
| FANCA | Involved in tolerance or repair of | NM_000135 |
| | DNA cross-links | |
| FANCB | Involved in tolerance or repair of | N/A |
| | DNA cross-links | |
| FANCC | Involved in tolerance or repair of | NM_000136 |
| | DNA cross-links | |
| FANCD | Involved in tolerance or repair of | N/A |
| | DNA cross-links | |
| FANCE | Involved in tolerance or repair of | NM_021922 |
| | DNA cross-links | |
| FANCF | Involved in tolerance or repair of | AF181994 |
| | DNA cross-links | |
| FANCG (XRCC9) | Involved in tolerance or repair of | NM_004629 |
| | DNA cross-links | |
| Other identified genes | | |
| with a suspected DNA | | |
| repair function | | |
| SNM1 (PS02) | DNA cross-link repair | D42045 |
| SNM1B | Related to SNM1 | AL137856 |
| SNM1C | Related to SNM1 | AA315885 |
| RPA4 | Similar to RPA2 | NM_013347 |
| ABH (ALKB) | Resistance to alkylation damage | X91992 |
| PNKP | Converts some DNA breaks to | NM_007254 |
| | ligatable ends | |
| Other conserved DNA | | |
| damage response genes | | |
| ATR | ATM- and PI-3K-like essential | NM_001184 |
| | kinase | |
| RAD1 (S. pombe) | PCNA-like DNA damage sensor | NM_002853 |
| homolog | | |
| RAD9 (S. pombe) | PCNA-like DNA damage sensor | NM_004584 |
| homolog | | |
| HUS1 (S. pombe) homolog | PCNA-like DNA damage sensor | NM_004507 |
| RAD17 (RAD24) | RFC-like DNA damage sensor | NM_002873 |
| TP53BP1 | BRCT protein | NM_005657 |
| CHEK1 | Effector kinase | NM_001274 |
| CHK2 (Rad53) | Effector kinase | NM_007194 |

TABLE 3

| Gene Name | Gene Title | Biological Activity |
|---|---|---|
| RFC2 | replication factor C (activator 1) 2, | DNA replication |
| | 40 kDa | |
| XRCC6 | X-ray repair complementing | DNA ligation /// DNA repair /// double-strand |
| | defective repair in Chinese | break repair via nonhomologous end-joining /// |
| | hamster cells 6 (Ku autoantigen, | DNA recombination /// positive regulation of |
| | 70 kDa) | transcription, DNA-dependent /// double-strand |
| | | break repair via nonhomologous end-joining /// |
| | | response to DNA damage stimulus /// DNA |
| | | recombination |
| APOBEC | apolipoprotein B mRNA editing | For all of APOBEC1, APOBEC2, |
| | enzyme, catalytic polypeptide-like | APOBEC3A-H, and APOBEC4, cytidine |
| | | deaminases. |
| POLD2 | polymerase (DNA directed), delta | DNA replication /// DNA replication |
| | 2, regulatory subunit 50 kDa | |
| PCNA | proliferating cell nuclear antigen | regulation of progression through cell cycle /// |
| | | DNA replication /// regulation of DNA |
| | | replication /// DNA repair /// cell proliferation /// |
| | | phosphoinositide-mediated signaling /// |
| | | DNA replication |
| RPA1 | replication protein A1, 70 kDa | DNA-dependent DNA replication /// DNA |
| | | repair /// DNA recombination /// DNA |
| | | replication |
| RPA1 | replication protein A1, 70 kDa | DNA-dependent DNA replication /// DNA |
| | | repair /// DNA recombination /// DNA |
| | | replication |
| RPA2 | replication protein A2, 32 kDa | DNA replication /// DNA-dependent DNA |
| | | replication |
| ERCC3 | excision repair cross- | DNA topological change /// transcription- |
| | complementing rodent repair | coupled nucleotide-excision repair /// |
| | deficiency, complementation | transcription /// regulation of transcription, |
| | group 3 (xeroderma pigmentosum | DNA-dependent /// transcription from RNA |
| | group B complementing) | polymerase II promoter /// induction of |
| | | apoptosis /// sensory perception of sound /// |
| | | DNA repair /// nucleotide-excision repair /// |
| | | response to DNA damage stimulus /// DNA |
| | | repair |
| UNG | uracil-DNA glycosylase | carbohydrate metabolism /// DNA repair /// |
| | | base-excision repair /// response to DNA |
| | | damage stimulus /// DNA repair /// DNA repair |
| ERCC5 | excision repair cross- | transcription-coupled nucleotide-excision repair /// |
| | complementing rodent repair | nucleotide-excision repair /// sensory |
| | deficiency, complementation | perception of sound /// DNA repair /// response |
| | group 5 (xeroderma pigmentosum, | to DNA damage stimulus /// nucleotide- |
| | complementation group G | excision repair |
| | (Cockayne syndrome)) | |
| MLH1 | mutL homolog 1, colon cancer, | mismatch repair /// cell cycle /// negative |
| | nonpolyposis type 2 (E. coli) | regulation of progression through cell cycle /// |
| | | DNA repair /// mismatch repair /// response to |
| | | DNA damage stimulus |
| LIG1 | ligase I, DNA, ATP-dependent | DNA replication /// DNA repair /// DNA |
| | | recombination /// cell cycle /// morphogenesis /// |
| | | cell division /// DNA repair /// response to |
| | | DNA damage stimulus /// DNA metabolism |
| NBN | nibrin | DNA damage checkpoint /// cell cycle |
| | | checkpoint /// double-strand break repair |
| NBN | nibrin | DNA damage checkpoint /// cell cycle |
| | | checkpoint /// double-strand break repair |
| NBN | nibrin | DNA damage checkpoint /// cell cycle |
| | | checkpoint /// double-strand break repair |
| MSH6 | mutS homolog 6 (E. coli) | mismatch repair /// DNA metabolism /// DNA |
| | | repair /// mismatch repair /// response to DNA |
| | | damage stimulus |
| POLD4 | polymerase (DNA-directed), delta | DNA replication /// DNA replication |
| | 4 | |
| RFC5 | replication factor C (activator 1) 5, | DNA replication /// DNA repair /// DNA |
| | 36.5 kDa | replication |
| RFC5 | replication factor C (activator 1) 5, | DNA replication /// DNA repair /// DNA |
| | 36.5 kDa | replication |
| DDB2 /// | damage-specific DNA binding | nucleotide-excision repair /// regulation of |
| LHX3 | protein 2, 48 kDa /// LIM | transcription, DNA-dependent /// organ |
| | homeobox 3 | morphogenesis /// DNA repair /// response to |
| | | DNA damage stimulus /// DNA repair /// |
| | | transcription /// regulation of transcription |
| POLD1 | polymerase (DNA directed), delta | DNA replication /// DNA repair /// response to |
| | 1, catalytic subunit 125 kDa | UV /// DNA replication |
| FANCG | Fanconi anemia, complementation | cell cycle checkpoint /// DNA repair /// DNA |
| | group G | repair /// response to DNA damage stimulus /// |
| | | regulation of progression through cell cycle |
| POLB | polymerase (DNA directed), beta | DNA-dependent DNA replication /// DNA |
| | | repair /// DNA replication /// DNA repair /// |
| | | response to DNA damage stimulus |
| XRCC1 | X-ray repair complementing | single strand break repair |
| | defective repair in Chinese | |
| | hamster cells 1 | |
| MPG | N-methylpurine-DNA glycosylase | base-excision repair /// DNA dealkylation /// |
| | | DNA repair /// base-excision repair /// response |
| | | to DNA damage stimulus |
| RFC2 | replication factor C (activator 1) 2, | DNA replication |
| | 40 kDa | |
| ERCC1 | excision repair cross- | nucleotide-excision repair /// morphogenesis /// |
| | complementing rodent repair | nucleotide-excision repair /// DNA repair /// |
| | deficiency, complementation | response to DNA damage stimulus |
| | group 1 (includes overlapping | |
| | antisense sequence) | |
| TDG | thymine-DNA glycosylase | carbohydrate metabolism /// base-excision |
| | | repair /// DNA repair /// response to DNA |
| | | damage stimulus |
| TDG | thymine-DNA glycosylase | carbohydrate metabolism /// base-excision |
| | | repair /// DNA repair /// response to DNA |
| | | damage stimulus |
| FANCA | Fanconi anemia, complementation | DNA repair /// protein complex assembly /// |
| | group A /// Fanconi anemia, | DNA repair /// response to DNA damage |
| | complementation group A | stimulus |
| RFC4 | replication factor C (activator 1) 4, | DNA replication /// DNA strand elongation /// |
| | 37 kDa | DNA repair /// phosphoinositide-mediated |
| | | signaling /// DNA replication |
| RFC3 | replication factor C (activator 1) 3, | DNA replication /// DNA strand elongation |
| | 38 kDa | |
| RFC3 | replication factor C (activator 1) 3, | DNA replication /// DNA strand elongation |
| | 38 kDa | |
| APEX2 | APEX nuclease | DNA repair /// response to DNA damage |
| | (apurinic/apyrimidinic | stimulus |
| | endonuclease) 2 | |
| RAD1 | RAD1 homolog (S. pombe) | DNA repair /// cell cycle checkpoint /// cell |
| | | cycle checkpoint /// DNA damage checkpoint /// |
| | | DNA repair /// response to DNA damage |
| | | stimulus /// meiotic prophase I |
| RAD1 | RAD1 homolog (S. pombe) | DNA repair /// cell cycle checkpoint /// cell |
| | | cycle checkpoint /// DNA damage checkpoint /// |
| | | DNA repair /// response to DNA damage |
| | | stimulus /// meiotic prophase I |
| BRCA1 | breast cancer 1, early onset | regulation of transcription from RNA |
| | | polymerase II promoter /// regulation of |
| | | transcription from RNA polymerase III |
| | | promoter /// DNA damage response, signal |
| | | transduction by p53 class mediator resulting in |
| | | transcription of p21 class mediator /// cell cycle /// |
| | | protein ubiquitination /// androgen receptor |
| | | signaling pathway /// regulation of cell |
| | | proliferation /// regulation of apoptosis /// |
| | | positive regulation of DNA repair /// negative |
| | | regulation of progression through cell cycle /// |
| | | positive regulation of transcription, DNA- |
| | | dependent /// negative regulation of centriole |
| | | replication /// DNA damage response, signal |
| | | transduction resulting in induction of apoptosis /// |
| | | DNA repair /// response to DNA damage |
| | | stimulus /// protein ubiquitination /// DNA |
| | | repair /// regulation of DNA repair /// apoptosis /// |
| | | response to DNA damage stimulus |
| EXO1 | exonuclease 1 | DNA repair /// DNA repair /// mismatch repair /// |
| | | DNA recombination |
| FEN1 | flap structure-specific | DNA replication /// double-strand break repair /// |
| | endonuclease 1 | UV protection /// phosphoinositide-mediated |
| | | signaling /// DNA repair /// DNA replication /// |
| | | DNA repair /// DNA repair |
| FEN1 | flap structure-specific | DNA replication /// double-strand break repair /// |
| | endonuclease 1 | UV protection /// phosphoinositide-mediated |
| | | signaling /// DNA repair /// DNA replication /// |
| | | DNA repair /// DNA repair |
| MLH3 | mutL homolog 3 (E. coli) | mismatch repair /// meiotic recombination /// |
| | | DNA repair /// mismatch repair /// response to |
| | | DNA damage stimulus /// mismatch repair |
| MGMT | O-6-methylguanine-DNA | DNA ligation /// DNA repair /// response to |
| | methyltransferase | DNA damage stimulus |
| RAD51 | RAD51 homolog (RecA homolog, | double-strand break repair via homologous |
| | E. coli) (S. cerevisiae) | recombination /// DNA unwinding during |
| | | replication /// DNA repair /// mitotic |
| | | recombination /// meiosis /// meiotic |
| | | recombination /// positive regulation of DNA |
| | | ligation /// protein homo-oligomerization /// |
| | | response to DNA damage stimulus /// DNA |
| | | metabolism /// DNA repair /// response to DNA |
| | | damage stimulus /// DNA repair /// DNA |
| | | recombination /// meiotic recombination /// |
| | | double-strand break repair via homologous |
| | | recombination /// DNA unwinding during |
| | | replication |
| RAD51 | RAD51 homolog (RecA homolog, | double-strand break repair via homologous |
| | E. coli) (S. cerevisiae) | recombination /// DNA unwinding during |
| | | replication /// DNA repair /// mitotic |
| | | recombination /// meiosis /// meiotic |
| | | recombination /// positive regulation of DNA |
| | | ligation /// protein homo-oligomerization /// |
| | | response to DNA damage stimulus /// DNA |
| | | metabolism /// DNA repair /// response to DNA |
| | | damage stimulus /// DNA repair /// DNA |
| | | recombination /// meiotic recombination /// |
| | | double-strand break repair via homologous |
| | | recombination /// DNA unwinding during |
| | | replication |
| XRCC4 | X-ray repair complementing | DNA repair /// double-strand break repair /// |
| | defective repair in Chinese | DNA recombination /// DNA recombination /// |
| | hamster cells 4 | response to DNA damage stimulus |
| XRCC4 | X-ray repair complementing | DNA repair /// double-strand break repair /// |
| | defective repair in Chinese | DNA recombination /// DNA recombination /// |
| | hamster cells 4 | response to DNA damage stimulus |
| RECQL | RecQ protein-like (DNA helicase | DNA repair /// DNA metabolism |
| | Q1-like) | |
| ERCC8 | excision repair cross- | DNA repair /// transcription /// regulation of |
| | complementing rodent repair | transcription, DNA-dependent /// sensory |
| | deficiency, complementation | perception of sound /// transcription-coupled |
| | group 8 | nucleotide-excision repair |
| FANCC | Fanconi anemia, complementation | DNA repair /// DNA repair /// protein complex |
| | group C | assembly /// response to DNA damage stimulus |
| OGG1 | 8-oxoguanine DNA glycosylase | carbohydrate metabolism /// base-excision |
| | | repair /// DNA repair /// base-excision repair /// |
| | | response to DNA damage stimulus /// DNA |
| | | repair |
| MRE11A | MRE11 meiotic recombination 11 | regulation of mitotic recombination /// double- |
| | homolog A (S. cerevisiae) | strand break repair via nonhomologous end- |
| | | joining /// telomerase-dependent telomere |
| | | maintenance /// meiosis /// meiotic |
| | | recombination /// DNA metabolism /// DNA |
| | | repair /// double-strand break repair /// response |
| | | to DNA damage stimulus /// DNA repair /// |
| | | double-strand break repair /// DNA |
| | | recombination |
| RAD52 | RAD52 homolog (S. cerevisiae) | double-strand break repair /// mitotic |
| | | recombination /// meiotic recombination /// |
| | | DNA repair /// DNA recombination /// response |
| | | to DNA damage stimulus |
| WRN | Werner syndrome | DNA metabolism /// aging |
| XPA | xeroderma pigmentosum, | nucleotide-excision repair /// DNA repair /// |
| | complementation group A | response to DNA damage stimulus /// DNA |
| | | repair /// nucleotide-excision repair |
| BEM | Bloom syndrome | DNA replication /// DNA repair /// DNA |
| | | recombination /// antimicrobial humoral |
| | | response (sensu Vertebrata) /// DNA |
| | | metabolism /// DNA replication |
| OGG1 | 8-oxoguanine DNA glycosylase | carbohydrate metabolism /// base-excision |
| | | repair /// DNA repair /// base-excision repair /// |
| | | response to DNA damage stimulus /// DNA |
| | | repair |
| MSH3 | mutS homolog 3 (E. coli) | mismatch repair /// DNA metabolism /// DNA |
| | | repair /// mismatch repair /// response to DNA |
| | | damage stimulus |
| POLE2 | polymerase (DNA directed), | DNA replication /// DNA repair /// DNA |
| | epsilon 2 (p59 subunit) | replication |
| RAD51C | RAD51 homolog C (S. cerevisiae) | DNA repair /// DNA recombination /// DNA |
| | | metabolism /// DNA repair /// DNA |
| | | recombination /// response to DNA damage |
| | | stimulus |
| LIG4 | ligase IV, DNA, ATP-dependent | single strand break repair /// DNA replication /// |
| | | DNA recombination /// cell cycle /// cell |
| | | division /// DNA repair /// response to DNA |
| | | damage stimulus |
| ERCC6 | excision repair cross- | DNA repair /// transcription /// regulation of |
| | complementing rodent repair | transcription, DNA-dependent /// transcription |
| | deficiency, complementation | from RNA polymerase II promoter /// sensory |
| | group 6 | perception of sound |
| LIG3 | ligase III, DNA, ATP-dependent | DNA replication /// DNA repair /// cell cycle /// |
| | | meiotic recombination /// spermatogenesis /// |
| | | cell division /// DNA repair /// DNA |
| | | recombination /// response to DNA damage |
| | | stimulus |
| RAD17 | RAD17 homolog (S. pombe) | DNA replication /// DNA repair /// cell cycle /// |
| | | response to DNA damage stimulus |
| XRCC2 | X-ray repair complementing | DNA repair /// DNA recombination /// meiosis /// |
| | defective repair in Chinese | DNA metabolism /// DNA repair /// response |
| | hamster cells 2 | to DNA damage stimulus |
| MUTYH | mutY homolog (E. coli) | carbohydrate metabolism /// base-excision |
| | | repair /// mismatch repair /// cell cycle /// |
| | | negative regulation of progression through cell |
| | | cycle /// DNA repair /// response to DNA |
| | | damage stimulus /// DNA repair |
| RFC1 | replication factor C (activator 1) 1, | DNA-dependent DNA replication /// |
| | 145 kDa /// replication factor C | transcription /// regulation of transcription, |
| | (activator 1) 1, 145 kDa | DNA-dependent /// telomerase-dependent |
| | | telomere maintenance /// DNA replication /// |
| | | DNA repair |
| RFC1 | replication factor C (activator 1) 1, | DNA-dependent DNA replication /// |
| | 145 kDa | transcription /// regulation of transcription, |
| | | DNA-dependent /// telomerase-dependent |
| | | telomere maintenance /// DNA replication /// |
| | | DNA repair |
| BRCA2 | breast cancer 2, early onset | regulation of progression through cell cycle /// |
| | | double-strand break repair via homologous |
| | | recombination /// DNA repair /// establishment |
| | | and/or maintenance of chromatin architecture /// |
| | | chromatin remodeling /// regulation of S phase |
| | | of mitotic cell cycle /// mitotic checkpoint /// |
| | | regulation of transcription /// response to DNA |
| | | damage stimulus |
| RAD50 | RAD50 homolog (S. cerevisiae) | regulation of mitotic recombination /// double- |
| | | strand break repair /// telomerase-dependent |
| | | telomere maintenance /// cell cycle /// meiosis /// |
| | | meiotic recombination /// chromosome |
| | | organization and biogenesis /// telomere |
| | | maintenance /// DNA repair /// response to |
| | | DNA damage stimulus /// DNA repair /// DNA |
| | | recombination |
| DDB1 | damage-specific DNA binding | nucleotide-excision repair /// ubiquitin cycle /// |
| | protein 1, 127 kDa | DNA repair /// response to DNA damage |
| | | stimulus /// DNA repair |
| XRCC5 | X-ray repair complementing | double-strand break repair via nonhomologous |
| | defective repair in Chinese | end-joining /// DNA recombination /// DNA |
| | hamster cells 5 (double-strand- | repair /// DNA recombination /// response to |
| | break rejoining; Ku autoantigen, | DNA damage stimulus /// double-strand break |
| | 80 kDa) | repair |
| XRCC5 | X-ray repair complementing | double-strand break repair via nonhomologous |
| | defective repair in Chinese | end-joining /// DNA recombination /// DNA |
| | hamster cells 5 (double-strand- | repair /// DNA recombination /// response to |
| | break rejoining; Ku autoantigen, | DNA damage stimulus /// double-strand break |
| | 80 kDa) | repair |
| PARP1 | poly (ADP-ribose) polymerase | DNA repair /// transcription from RNA |
| | family, member 1 | polymerase II promoter /// protein amino acid |
| | | ADP-ribosylation /// DNA metabolism /// DNA |
| | | repair /// protein amino acid ADP-ribosylation /// |
| | | response to DNA damage stimulus |
| POLE3 | polymerase (DNA directed), | DNA replication |
| | epsilon 3 (p17 subunit) | |
| RFC1 | replication factor C (activator 1) 1, | DNA-dependent DNA replication /// |
| | 145 kDa | transcription /// regulation of transcription, |
| | | DNA-dependent /// telomerase-dependent |
| | | telomere maintenance /// DNA replication /// |
| | | DNA repair |
| RAD50 | RAD50 homolog (S. cerevisiae) | regulation of mitotic recombination /// double- |
| | | strand break repair /// telomerase-dependent |
| | | telomere maintenance /// cell cycle /// meiosis /// |
| | | meiotic recombination /// chromosome |
| | | organization and biogenesis /// telomere |
| | | maintenance /// DNA repair /// response to |
| | | DNA damage stimulus /// DNA repair /// DNA |
| | | recombination |
| XPC | xeroderma pigmentosum, | nucleotide-excision repair /// DNA repair /// |
| | complementation group C | nucleotide-excision repair /// response to DNA |
| | | damage stimulus /// DNA repair |
| MSH2 | mutS homolog 2, colon cancer, | mismatch repair /// post-replication repair /// |
| | nonpolyposis type 1 (E. coli) | cell cycle /// negative regulation of progression |
| | | through cell cycle /// DNA metabolism /// DNA |
| | | repair /// mismatch repair /// response to DNA |
| | | damage stimulus /// DNA repair |
| RPA3 | replication protein A3, 14 kDa | DNA replication /// DNA repair /// DNA |
| | | replication |
| MBD4 | methyl-CpG binding domain | base-excision repair /// DNA repair /// response |
| | protein 4 | to DNA damage stimulus /// DNA repair |
| MBD4 | methyl-CpG binding domain | base-excision repair /// DNA repair /// response |
| | protein 4 | to DNA damage stimulus /// DNA repair |
| NTHL1 | nth endonuclease III-like 1 | carbohydrate metabolism /// base-excision |
| | (E. coli) | repair /// nucleotide-excision repair, DNA |
| | | incision, 5′-to lesion /// DNA repair /// response |
| | | to DNA damage stimulus |
| PMS2 /// | PMS2 post-meiotic segregation | mismatch repair /// cell cycle /// negative |
| PMS2CL | increased 2 (S. cerevisiae) /// | regulation of progression through cell cycle /// |
| | PMS2-C terminal-like | DNA repair /// mismatch repair /// response to |
| | | DNA damage stimulus /// mismatch repair |
| RAD51C | RAD51 homolog C (S. cerevisiae) | DNA repair /// DNA recombination /// DNA |
| | | metabolism /// DNA repair /// DNA |
| | | recombination /// response to DNA damage |
| | | stimulus |
| UNG2 | uracil-DNA glycosylase 2 | regulation of progression through cell cycle /// |
| | | carbohydrate metabolism /// base-excision |
| | | repair /// DNA repair /// response to DNA |
| | | damage stimulus |
| APEX1 | APEX nuclease (multifunctional | base-excision repair /// transcription from RNA |
| | DNA repair enzyme) 1 | polymerase II promoter /// regulation of DNA |
| | | binding /// DNA repair /// response to DNA |
| | | damage stimulus |
| ERCC4 | excision repair cross- | nucleotide-excision repair /// nucleotide- |
| | complementing rodent repair | excision repair /// DNA metabolism /// DNA |
| | deficiency, complementation | repair /// response to DNA damage stimulus |
| | group 4 | |
| RAD1 | RAD1 homolog (S. pombe) | DNA repair /// cell cycle checkpoint /// cell |
| | | cycle checkpoint /// DNA damage checkpoint /// |
| | | DNA repair /// response to DNA damage |
| | | stimulus /// meiotic prophase I |
| RECQL5 | RecQ protein-like 5 | DNA repair /// DNA metabolism /// DNA |
| | | metabolism |
| MSH5 | mutS homolog 5 (E. coli) | DNA metabolism /// mismatch repair /// |
| | | mismatch repair /// meiosis /// meiotic |
| | | recombination /// meiotic prophase II /// meiosis |
| RECQL | RecQ protein-like (DNA helicase | DNA repair /// DNA metabolism |
| | Q1-like) | |
| RAD52 | RAD52 homolog (S. cerevisiae) | double-strand break repair /// mitotic |
| | | recombination /// meiotic recombination /// |
| | | DNA repair /// DNA recombination /// response |
| | | to DNA damage stimulus |
| XRCC4 | X-ray repair complementing | DNA repair /// double-strand break repair /// |
| | defective repair in Chinese | DNA recombination /// DNA recombination /// |
| | hamster cells 4 | response to DNA damage stimulus |
| XRCC4 | X-ray repair complementing | DNA repair /// double-strand break repair /// |
| | defective repair in Chinese | DNA recombination /// DNA recombination /// |
| | hamster cells 4 | response to DNA damage stimulus |
| RAD17 | RAD17 homolog (S. pombe) | DNA replication /// DNA repair /// cell cycle /// |
| | | response to DNA damage stimulus |
| MSH3 | mutS homolog 3 (E. coli) | mismatch repair /// DNA metabolism /// DNA |
| | | repair /// mismatch repair /// response to DNA |
| | | damage stimulus |
| MRE11A | MRE11 meiotic recombination 11 | regulation of mitotic recombination /// double- |
| | homolog A (S. cerevisiae) | strand break repair via nonhomologous end- |
| | | joining /// telomerase-dependent telomere |
| | | maintenance /// meiosis /// meiotic |
| | | recombination /// DNA metabolism /// DNA |
| | | repair /// double-strand break repair /// response |
| | | to DNA damage stimulus /// DNA repair /// |
| | | double-strand break repair /// DNA |
| | | recombination |
| MSH6 | mutS homolog 6 (E. coli) | mismatch repair /// DNA metabolism /// DNA |
| | | repair /// mismatch repair /// response to DNA |
| | | damage stimulus |
| MSH6 | mutS homolog 6 (E. coli) | mismatch repair /// DNA metabolism /// DNA |
| | | repair /// mismatch repair /// response to DNA |
| | | damage stimulus |
| RECQL5 | RecQ protein-like 5 | DNA repair /// DNA metabolism /// DNA |
| | | metabolism |
| BRCA1 | breast cancer 1, early onset | regulation of transcription from RNA |
| | | polymerase II promoter /// regulation of |
| | | transcription from RNA polymerase III |
| | | promoter /// DNA damage response, signal |
| | | transduction by p53 class mediator resulting in |
| | | transcription of p21 class mediator /// cell cycle /// |
| | | protein ubiquitination /// androgen receptor |
| | | signaling pathway /// regulation of cell |
| | | proliferation /// regulation of apoptosis /// |
| | | positive regulation of DNA repair /// negative |
| | | regulation of progression through cell cycle /// |
| | | positive regulation of transcription, DNA- |
| | | dependent /// negative regulation of centriole |
| | | replication /// DNA damage response, signal |
| | | transduction resulting in induction of apoptosis /// |
| | | DNA repair /// response to DNA damage |
| | | stimulus /// protein ubiquitination /// DNA |
| | | repair /// regulation of DNA repair /// apoptosis /// |
| | | response to DNA damage stimulus |
| RAD52 | RAD52 homolog (S. cerevisiae) | double-strand break repair /// mitotic |
| | | recombination /// meiotic recombination /// |
| | | DNA repair /// DNA recombination /// response |
| | | to DNA damage stimulus |
| POLD3 | polymerase (DNA-directed), delta | DNA synthesis during DNA repair /// mismatch |
| | 3, accessory subunit | repair /// DNA replication |
| MSH5 | mutS homolog 5 (E. coli) | DNA metabolism /// mismatch repair /// |
| | | mismatch repair /// meiosis /// meiotic |
| | | recombination /// meiotic prophase II /// meiosis |
| ERCC2 | excision repair cross- | transcription-coupled nucleotide-excision repair /// |
| | complementing rodent repair | transcription /// regulation of transcription, |
| | deficiency, complementation | DNA-dependent /// transcription from RNA |
| | group 2 (xeroderma pigmentosum | polymerase II promoter /// induction of |
| | D) | apoptosis /// sensory perception of sound /// |
| | | nucleobase, nucleoside, nucleotide and nucleic |
| | | acid metabolism /// nucleotide-excision repair |
| RECQL4 | RecQ protein-like 4 | DNA repair /// development /// DNA |
| | | metabolism |
| PMS1 | PMS1 post-meiotic segregation | mismatch repair /// regulation of transcription, |
| | increased 1 (S. cerevisiae) | DNA-dependent /// cell cycle /// negative |
| | | regulation of progression through cell cycle /// |
| | | mismatch repair /// DNA repair /// response to |
| | | DNA damage stimulus |
| ZFP276 | zinc finger protein 276 homolog | transcription /// regulation of transcription, |
| | (mouse) | DNA-dependent |
| MBD4 | methyl-CpG binding domain | base-excision repair /// DNA repair /// response |
| | protein 4 | to DNA damage stimulus /// DNA repair |
| MBD4 | methyl-CpG binding domain | base-excision repair /// DNA repair /// response |
| | protein 4 | to DNA damage stimulus /// DNA repair |
| MLH3 | mutL homolog 3 (E. coli) | mismatch repair /// meiotic recombination /// |
| | | DNA repair /// mismatch repair /// response to |
| | | DNA damage stimulus /// mismatch repair |
| FANCA | Fanconi anemia, complementation | DNA repair /// protein complex assembly /// |
| | group A | DNA repair /// response to DNA damage |
| | | stimulus |
| POLE | polymerase (DNA directed), | DNA replication /// DNA repair /// DNA |
| | epsilon | replication /// response to DNA damage |
| | | stimulus |
| XRCC3 | X-ray repair complementing | DNA repair /// DNA recombination /// DNA |
| | defective repair in Chinese | metabolism /// DNA repair /// DNA |
| | hamster cells 3 | recombination /// response to DNA damage |
| | | stimulus /// response to DNA damage stimulus |
| MLH3 | mutL homolog 3 (E. coli) | mismatch repair /// meiotic recombination /// |
| | | DNA repair /// mismatch repair /// response to |
| | | DNA damage stimulus /// mismatch repair |
| NBN | nibrin | DNA damage checkpoint /// cell cycle |
| | | checkpoint /// double-strand break repair |
| SMUG1 | single-strand selective | carbohydrate metabolism /// DNA repair /// |
| | monofunctional uracil DNA | response to DNA damage stimulus |
| | glycosylase | |
| FANCF | Fanconi anemia, complementation | DNA repair /// response to DNA damage |
| | group F | stimulus |
| NEIL1 | nei endonuclease VIII-like 1 | carbohydrate metabolism /// DNA repair /// |
| | (E. coli) | response to DNA damage stimulus |
| FANCE | Fanconi anemia, complementation | DNA repair /// response to DNA damage |
| | group E | stimulus |
| MSH5 | mutS homolog 5 (E. coli) | DNA metabolism /// mismatch repair /// |
| | | mismatch repair /// meiosis /// meiotic |
| | | recombination /// meiotic prophase II /// meiosis |
| RECQL5 | RecQ protein-like 5 | DNA repair /// DNA metabolism /// DNA |
| | | metabolism |

In yet another example, cfDNA/cfRNA may be derived from a gene not associated with a disease (e.g., housekeeping genes), which include those related to transcription factors (e.g., ATF1, ATF2, ATF4, ATF6, ATF7, ATFIP, BTF3, E2F4, ERH, HMGB1, ILF2, IER2, JUND, TCEB2, etc.), repressors (e.g., PUF60), RNA splicing (e.g., BAT1, HNRPD, HNRPK, PABPN1, SRSF3, etc.), translation factors (EIF1, EIF1AD, EIF1B, EIF2A, EIF2AK1, EIF2AK3, EIF2AK4, EIF2B2, EIF2B3, EIF2B4, EIF2S2, EIF3A, etc.), tRNA synthetases (e.g., AARS, CARS, DARS, FARS, GARS, HARS, IARS, KARS, MARS, etc.), RNA binding protein (e.g., ELAVL1, etc.), ribosomal proteins (e.g., RPL5, RPL8, RPL9, RPL10, RPL11, RPL14, RPL25, etc.), mitochondrial ribosomal proteins (e.g., MRPL9, MRPL1, MRPL10, MRPL11, MRPL12, MRPL13, MRPL14, etc.), RNA polymerase (e.g., POLR1C, POLR1D, POLR1E, POLR2A, POLR2B, POLR2C, POLR2D, POLR3C, etc.), protein processing (e.g., PPID, PPI3, PPIF, CANX, CAPN1, NACA, PFDN2, SNX2, SS41, SUMO1, etc.), heat shock proteins (e.g., HSPA4, HSPA5, HSBP1, etc.), histone (e.g., HIST1HSBC, H1FX, etc.), cell cycle (e.g., ARHGAP35, RAB10, RAB11A, CCNY, CCNL, PPP1CA, RAD1, RAD17, etc.), carbohydrate metabolism (e.g., ALDOA, GSK3A, PGK1, PGAM5, etc.), lipid metabolism (e.g., HADHA), citric acid cycle (e.g., SDHA, SDHB, etc.), amino acid metabolism (e.g., COMT, etc.), NADH dehydrogenase (e.g., NDUFA2, etc.), cytochrome c oxidase (e.g., COX5B, COX8, COX11, etc.), ATPase (e.g. ATP2C1, ATP5F1, etc.), lysosome (e.g., CTSD, CSTB, LAMP1, etc.), proteasome (e.g., PSMA1, UBA1, etc.), cytoskeletal proteins (e.g., ANXA6, ARPC2, etc.), and organelle synthesis (e.g., BLOC1S1, AP2A1, etc.). It is further contemplated that cfDNA/cfRNA may be derived from genes that are specific to a diseased cell or organ (e.g., PCA3, PSA, etc.), or that are commonly found in cancer patients, including various mutations in KRAS (e.g., G12V, G12D, G12C, etc.) or BRAF (e.g., V600E, etc.).

It is also contemplated that ctDNA/ctRNA or cfRNA may present in modified forms or different isoforms. For example, the ctDNA may be present in methylated or hydroxyl methylated, and the methylation level of some genes (e.g., GSTP1, p16, APC, etc.) may be a hallmark of specific types of cancer (e.g., colorectal cancer, etc.). The ctRNA may be present in a plurality of isoforms (e.g., splicing variants, etc.) that may be associated with different cell types and/or location. Preferably, different isoforms of ctRNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, mRNA encoding HMGB1 are present in 18 different alternative splicing variants and 2 unspliced forms. Those isoforms are expected to express in different tissues/locations of the patient's body (e.g., isoform A is specific to prostate, isoform B is specific to brain, isoform C is specific to spleen, etc.). Thus, in these embodiments, identifying the isoforms of ctRNA in the patient's bodily fluid can provide information on the origin (e.g., cell type, tissue type, etc.) of the ctRNA.

Alternatively or additionally, the inventors contemplate ctRNA may include regulatory noncoding RNA (e.g., microRNA, small interfering RNA, long non-coding RNA (1ncRNA)), which quantities and/or isoforms (or subtypes) can vary and fluctuate by presence of a tumor or immune response against the tumor. Without wishing to be bound by any specific theory, varied expression of regulatory noncoding RNA in a cancer patient's bodily fluid may due to genetic modification of the cancer cell (e.g., deletion, translocation of parts of a chromosome, etc.), and/or inflammations at the cancer tissue by immune system (e.g., regulation of miR-29 family by activation of interferon signaling and/or virus infection, etc.). Thus, in some embodiments, the ctRNA can be a regulatory noncoding RNA that modulates expression (e.g., downregulates, silences, etc.) of mRNA encoding a cancer-related protein or an inflammation-related protein (e.g., HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, hTERT, etc.).

It is also contemplated that some cell free regulatory noncoding RNA may be present in a plurality of isoforms or members (e.g., members of miR-29 family, etc.) that may be associated with different cell types and/or location. Preferably, different isoforms or members of regulatory noncoding RNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, higher expression level of miR-155 in the bodily fluid can be associated with the presence of breast tumor, and the reduced expression level of miR-155 can be associated with reduced size of breast tumor. Thus, in these embodiments, identifying the isoforms of cell free regulatory noncoding RNA in the patient's bodily fluid can provide information on the origin (e.g., cell type, tissue type, etc.) of the cell free regulatory noncoding RNA.

Thus, it should be appreciated that one or more desired cfDNA/cfRNA may be selected for a particular disease (e.g., different types of tumor or cancer, etc.), disease stage (early phase, metastasis, etc.), disease status (e.g., endothelial-mesenchymal transition, immune suppression, loss of immune response, change of molecular profile of tumor cells, change in clonality, etc.), specific mutation, or even on the basis of personal mutational profiles or presence of expressed neoepitopes. Alternatively, where discovery or scanning for new mutations or changes in expression of a particular gene is desired, real time quantitative PCR may be replaced by or added with RNAseq to so cover at least part of a patient transcriptome. Moreover, it should be appreciated that analysis can be performed static or over a time course with repeated sampling to obtain a dynamic picture without the need for biopsy of the tumor or a metastasis.

Once cfDNA/cfRNA is isolated, various types of omics data can be obtained using any suitable methods. DNA sequence data will not only include the presence or absence of a gene that is associated with cancer or inflammation, but also take into account mutation data where the gene is mutated, the copy number (e.g., to identify duplication, loss of allele or heterozygosity), and epigenetic status (e.g., methylation, histone phosphorylation, nucleosome positioning, etc.). With respect to RNA sequence data it should be noted that contemplated RNA sequence data include mRNA sequence data, splice variant data, polyadenylation information, etc. Moreover, it is generally preferred that the RNA sequence data also include a metric for the transcription strength (e.g., number of transcripts of a damage repair gene per million total transcripts, number of transcripts of a damage repair gene per total number of transcripts for all damage repair genes, number of transcripts of a damage repair gene per number of transcripts for actin or other household gene RNA, etc.), and for the transcript stability (e.g., a length of poly A tail, etc.).

With respect to the transcription strength (expression level), transcription strength of the cfRNA can be examined by quantifying the ctRNA or cfRNA. Quantification of cfRNA can be performed in numerous manners, however, expression of analytes is preferably measured by quantitative real-time RT-PCR of cfRNA using primers specific for each gene. For example, amplification can be performed using an assay in a 10 μL reaction mix containing 2 μL cfRNA, primers, and probe. mRNA of α-actin or (β-actin can be used as an internal control for the input level of cfRNA. A standard curve of samples with known concentrations of each analyte was included in each PCR plate as well as positive and negative controls for each gene. Test samples were identified by scanning the 2D barcode on the matrix tubes containing the nucleic acids. Delta Ct (dCT) was calculated from the Ct value derived from quantitative PCR (qPCR) amplification for each analyte subtracted by the Ct value of actin for each individual patient's blood sample. Relative expression of patient specimens is calculated using a standard curve of delta Cts of serial dilutions of Universal Human Reference RNA or another control known to express the gene of interest set at a gene expression value of 10 or a suitable whole number allowing for a range of patient sample results for the specific to be resulted in the range of approximately 1 to 1000 (when the delta CTs were plotted against the log concentration of each analyte). Alternatively and/or additionally, Delta Cts vs. log₁₀Relative Gene Expression (standard curves) for each gene test can be captured over hundreds of PCR plates of reactions (historical reactions). A linear regression analysis can be performed for each assays and used to calculate gene expression from a single point from the original standard curve going forward.

Alternatively or additionally, where discovery or scanning for new mutations or changes in expression of a particular gene is desired, real time quantitative PCR may be replaced by or added with RNAseq to so cover at least part of a patient transcriptome. Moreover, it should be appreciated that analysis can be performed static or over a time course with repeated sampling to obtain a dynamic picture without the need for biopsy of the tumor or a metastasis. Thus, in addition to RNA quantification, RNA sequencing of the cfRNA (directly or via reverse transcription) may be performed to verify identity and/or identify post-transcriptional modifications, splice variations, and/or RNA editing. To that end, sequence information may be compared to prior RNA sequences of the same patient (of another patient, or a reference RNA), preferably using synchronous location guided analysis (e.g., using BAMBAM as described in US Pat. Pub. No. 2012/0059670 and/or US2012/0066001, etc.). Such analysis is particularly advantageous as such identified mutations can be filtered for neoepitopes that are unique to the patient, presented in the MHC I and/or II complex of the patient, and as such serve as therapeutic target. Moreover, suitable mutations may also be further characterized using a pathway model and the patient- and tumor-specific mutation to infer a physiological parameter of the tumor. For example, especially suitable pathway models include PARADIGM (see e.g., WO 2011/139345, WO 2013/062505) and similar models (see e.g., WO 2017/033154). Moreover, suitable mutations may also be unique to a sub-population of cancer cells. Thus, mutations may be selected based on the patient and specific tumor (and even metastasis), on the suitability as therapeutic target, type of gene (e.g., cancer driver gene), and affected function of the gene product encoded by the gene with the mutation.

Moreover, the inventors contemplate that multiple types of cfDNA and/or cfRNA can be isolated, detected and/or quantified from the same bodily fluid sample of the patient such that the relationship or association among the mutation, quantity, and/or subtypes of multiple cfDNA and/or cfRNA can be determined for further analysis. Thus, in one embodiment, from a single bodily fluid sample or from a plurality of bodily fluid samples obtained in a substantially similar time points, from a patient, multiple cfRNA species can be detected and quantified. In this embodiment, it is especially preferred that at least some of the cfRNA measurements are specific with respect to a cancer associated nucleic acid.

Consequently, such obtained omics data information of cfDNA/cfRNA of one or more gene can be used for diagnosis of tumor, monitoring of prognosis of the tumor, monitoring the effectiveness of treatment provided to the patients, evaluating a treatment regime based on a likelihood of success of the treatment regime, and even as discovery tool that allows repeated and non-invasive sampling of a patient.

For example, early detection of cancer, regardless specific anatomical or molecular type of tumor, can be achieved by measuring overall quantity of ctDNAs and/or ctRNAs in the sample of the patient's bodily fluid (as e.g., described in International Patent Application PCT/US18/22747, incorporated by reference herein). It is contemplated that presence of cancer in the patient can be assumed or inferred when overall cfDNA and/or cfRNA quantity reaches a particular or predetermined threshold. The predetermined threshold of cfDNA and/or cfRNA quantity can be determined by measuring overall cfDNA and/or cfRNA quantity from a plurality of healthy individuals in a similar physical condition (e.g., ethnicity, gender, age, other predisposed genetic or disease condition, etc.).

For example, predetermined threshold of cfDNA and/or cfRNA quantity is at least 20%, at least 30%, at least 40%, at least 50% more than the average or median number of cfDNA and/or cfRNA quantity of healthy individual. It should be appreciated that such approach to detect tumor early can be performed without a priori knowledge on anatomical or molecular characteristics or tumor, or even the presence of the tumor. To further obtain cancer specific information and/or information about the status of the immune system, additional cfRNA markers may be detected and/or quantified. Most typically, such additional cfRNA markers will include cfRNA encoding one or more oncogenes as described above and/or one or more cfRNA encoding a protein that is associated with immune suppression or other immune evading mechanism. Among other markers in such use, particularly contemplated cfRNAs include those encoding MUC1, MICA, brachyury, and/or PD-L1.

The inventors further contemplate that once the tumor is identified or detected, the prognosis of the tumor can be monitored by monitoring the types and/or quantity of cfDNAs and/or cfRNAs in various time points. As described, a patient- and tumor-specific mutation is identified in a gene of a tumor of the patient. Once identified, cfDNAs and/or cfRNAs, at least one of which comprises the patient- and tumor-specific mutation, are isolated from a bodily fluid of the patient (typically whole blood, plasma, serum), and then the mutation, quantity, and/or subtype of cfDNAs and/or cfRNAs are detected and/or quantified. The inventors contemplate that the mutation, quantity, and/or subtype of cfDNAs and/or cfRNAs detected from the patient's bodily fluid can be a strong indicator of the state, size, and location of the tumor. For example, increased quantity of cfDNAs and/or cfRNAs having a patient- and tumor-specific mutation can be an indicator of increased tumor cell lysis upon immune response against the tumor cell and/or increased numbers of tumor cells having the mutation. In another example, increased ratio of cfRNA over cfDNA having the patient- and tumor-specific mutation (where cfRNA and cfDNA are derived from the same gene having the mutation) may indicate that such patient- and tumor-specific mutation may cause increased transcription of the mutated gene to potentially trigger tumorigenesis or affects the tumor cell function (e.g., immune-resistance, related to metastasis, etc.). In still another example, increased quantity of a ctRNA having a patient- and tumor-specific mutation along with increased quantity of another ctRNA (or non-tumor related cfRNA) may indicate that the another ctRNA may be in the same pathway with the ctRNA having a patient- and tumor-specific mutation such that the expression or activity of two ctRNA (or a ctRNA and a cfRNA) may be correlated (e.g., co-regulated, one affect another, one is upstream of another in the pathway, etc.).

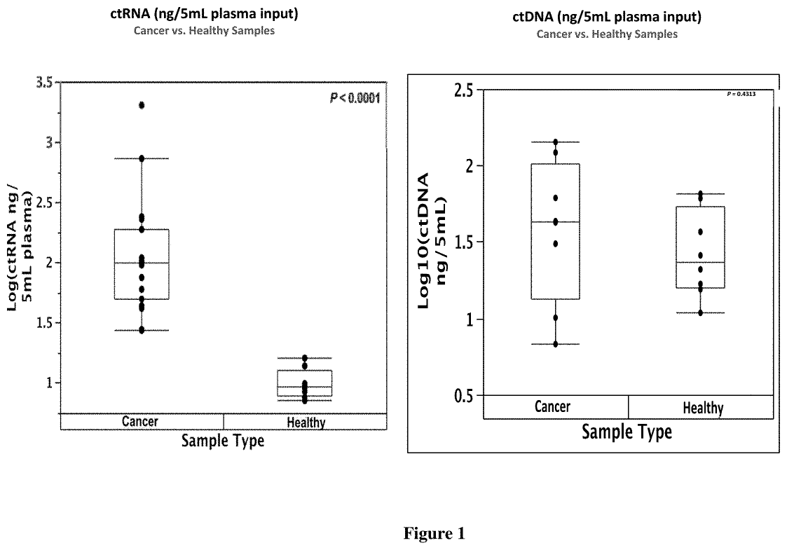

With regard to ctDNA, it should be noted that the accuracy of ctDNA in diagnostic tests has been in question since its adoption as a diagnostic tool for cancer. Issues with unusually high false positive rates must be addressed when relying on ctDNA in monitoring disease progression, but especially when considering the use of ctDNA in prediction of disease existence. As shown in FIG. 1, healthy individuals produce similar amounts of total ctDNA as cancer patients, however, levels of total cfRNA (e.g., as determined by quantitation using beta actin) are significantly low in healthy individuals. Moreover, when cfRNA isolation protocols were performed under conditions that did not lead to substantial cell lysis, the levels of total cfRNA were significantly different between cancer patients and healthy individuals. Indeed, there was no overlap between the groups of healthy individuals thereby allowing the cancer patients to be distinguished by their total cfRNA levels. Conversely, there was overlap between the levels of ctDNA in cancer patients and healthy individuals. Therefore ctDNA could not distinguish between these two groups. In further contemplated methods, it should be appreciated that where total cfRNA is isolated, cfDNA may be removed and/or degraded using appropriate DNAses (e.g., using on-column digestion of DNA). Likewise, where ctDNA is isolated, cfRNA may be removed and/or degraded using appropriate RNAses. Moreover, the linear detection range for cfRNA (here: PD-L1) was significant when isolation protocols were performed under conditions that did not lead to substantial cell lysis

Further, types and/or quantities of cfDNAs and/or cfRNAs can indicate the prognosis of the tumor, presence or progress of metastasis, possibility of metastasis, presence of cancer stem cells, presence of immune suppressive tumor microenvironment, increased or decreased immune cell activity or toxicity against tumor cells, or any cellular, molecular, anatomical, or biochemical changes in the tumor or around the tumor that results in change in cfDNA/cfRNA identity or expression, can be monitored by monitoring the types and/or quantity of cfDNAs and/or cfRNAs in various time points.

For example, contemplated analyses will include tests for analytes that are indicative of sternness of a cancer or cancer cell and/or for analytes that are indicative of epithelial to mesenchymal transition (EMT). Among other suitable analytes, cfRNA and/or cfDNA encoding all or a portion of DCC, UNC5A, and/or Netrin may be detected to identify cancer stem cell characteristics in one or more cancer cells. Likewise, cfRNA and/or cfDNA encoding all or a portion of IL-8, CXCR1, and/or CXCR2 may be detected to identify predisposition to the EMT. It should be appreciated that these exemplary analytes are physiologically ‘downstream’ of brachyury during development and may significantly contribute to the EMT, a role well assigned to brachyury. Thus, brachyury is also deemed particularly suitable for use herein, especially in conjunction with the above exemplary analytes. Advantageously, a combination of a drug targeting the netrin nexus may have significant therapeutic (synergistic) effect with drugs targeting brachyury (e.g., using cancer viral or yeast vaccines that target brachyury). Viewed form another perspective, diagnostic methods targeting the above exemplary analytes will identify potential for EMT and thus metastasis and resistance to conventional therapy (as cells having undergone EMT are often resistant to chemotherapies). In addition, and with further focus on IL-8/CXCR1/CXCR2, it should be appreciated that such analytes are also indicative of an immune-inhibitory mechanism employed by cancer cells. For example, CXCR2 ligands (e.g., CXCL1, CXCL2, CXCL5, and IL-8) attract myeloid derived suppressor cells (MDSC), which are immune inhibitory. CXCR2 is expressed on most of circulating MDSCs and is prerequisite for MDSCs to be recruited to tumor microenvironment.

In some embodiments, cfRNA and/or cfDNA of at least two distinct genes can be detected and analyzed to determine the status of tumor. Such two distinct genes may be related to a common target molecule (e.g., a signaling molecule that is activated by proteins encoded by two distinct genes, etc.), may be in the same signaling pathway, may be affected by a common upstream molecule (e.g., activated by phosphorylation by same type of kinase, etc.), or affected by the same physiological environment (e.g., immune suppressive environment, etc.). Thus, the cfRNA and/or cfDNA of at least two distinct genes may be derived from the same cell or same types of cell (e.g., same type of tumor cell, etc.), or from different cell types (e.g., one cfRNA and/or cfDNA is derived from a tumor cell and another cfRNA and/or cfDNA is derived from an immune competent cell or suppressive immune cell (e.g., MDSC cells, etc.) in the tumor microenvironment, etc.).

It is contemplated that various relationships between cfRNA and/or cfDNA of at least two distinct genes can be determined to associate with the cancer status. For example, absolute quantities or sum of absolute quantities (normalized with cfRNA of housekeeping gene, etc.) of cfRNAs of CXCR1 and CXCR2 can be associated with presence and/or development of immune-suppressive tumor microenvironment. In such example, the presence immune-suppressive tumor microenvironment or rapid development of immune-suppressive tumor microenvironment can be determined if the sum of CXCR1 and CXCR2 cfRNA quantities is determined above the pre-determined quantity threshold (as an absolute quantity or percentage increase compared to healthy individuals, etc.). In another example, a ratio of cfRNAs of two distinct genes can be associated with presence and/or development of immune-suppressive tumor microenvironment. Such example may include a ratio of cfRNAs of FoxP3 (a regulatory T cell marker) and cfRNAs of Ag 1 (Sca-1, which is upregulated upon activation of NK cells), and the presence and/or development of immune-suppressive tumor microenvironment can be determined if the ratio between the cfRNAs of FoxP3 and Ag1 is at least 0.5, at least 1, at least 2, at least 3, at least 5, or at least 10. In still other example, a sum or ratio of cfRNAs of two distinct genes can be associated with presence and/or development of EMT or cancer cell sternness. Such example may include the sum of cfRNAs of TGF-β1 and FOXC2 that may reflect the presence and/or development of EMT or cancer cell sternness when the sum is above the predetermined threshold (as an absolute quantity or percentage increase compared to healthy individuals, etc.). Such example may also include the ratio of cfRNAs of TGF-β1 and E-cadherin, that may reflect the presence and/or development of EMT or cancer cell stemness when the ratio is above the predetermined threshold (e.g., at least 0.5, at least 1, at least 2, at least 3, at least 5, or at least 10, etc.).

Additionally and/or alternatively, the inventors contemplate that cfDNAs from at least one gene can be further identified and analyzed to determine the cancer status. For example, cfDNA may be derived from a gene encoding zinc finger E-box binding homeobox transcription factor 1 (Zeb1), which may include one or more mutation in the gene to alter its sensitivity to EGFR inhibitors. In such example, the nucleic acid sequence analysis of cfDNA derived from ZEB1 in addition to the expression level of cfRNA of ZEB1 can be used together to determine the cancer status. For example, co-existence of a mutation in cfDNA derived from ZEB1 (whether the mutation is known mutation for EMT or not) and an increased expression of cfRNA of ZEB1 may be strongly associated with the presence and/or development of EMT or cancer stemness. In some embodiments, the number and/or location of the mutation and the level of increased expression can be considered as independent factors and/or as having same weight to determine the presence and/or development of EMT or cancer stemness. In other embodiments, the number, type, and/or location of the mutation and the level of increased expression may be given different weight (e.g., 30% increase of cfRNA level weighs at least twice higher than a presence single point mutation in the exon of ZEB1, a missense mutation in the exon of ZEB1 weighs at least 50% higher than 10% increase of ZEB1 cfRNA level, etc.).

Additionally, in some embodiments, the results of cfDNA/cfRNA analysis can be supplemented with identification and/or quantification of a peptide or a protein in the sample of the bodily fluid. Preferably, the peptide or a protein may be any secreted peptides from a tumor cell, an immune cell, or any other cells in the tumor microenvironment, which includes, but not limited to any type of cytokines (e.g., IL-1, IL-2, IL-4, IL-5, IL-9, IL-10, IL-13, IL-17, IL-22, IL-25, IL-30, IL-33, IFN-t, IFN-γ, etc.), chemokines (e.g., CCL2, CXCL14, CD40L, CCL2, CCL1, CCL22, CCL17, CXCR3, CXCL9, CXCL10, CXCL11, CXCL14, CXCR4, etc.), a receptor ligand (e.g., NKG2D ligands such as MICA, etc.). For example, NKD2D ligands (and especially soluble NKG2D ligands such as MICA, MICB, MBLL, and ULBP1-6) are known to reduce cytotoxic activity of NK cells and CTLs, and detection and/or quantification of ctRNA encoding NKG2D ligands (and especially soluble NKG2D ligands), and the quantity of soluble NKG2D may reflect the immune suppressive state of the tumor microenvironment, which may support the increase expression level of cfRNAs of FoxP3 and/or decreased expression level of Ag1. For example, a soluble and/or exosomal membrane bound NKG2D ligands on a protein level. may be detected in a large variety of methods, and especially contemplated methods include ELISA assays and mass spec based assays, which may provide additional information as to potential immune suppression that is due to downregulation of NKG2D on NK and T-cells.

Similarly, and as discussed in more detail below, other ctRNA that encode various immune modulatory factors, including PD-1L are also deemed suitable. Suitable ctRNA molecules may also encode proteins that indirectly down-regulate an anti-tumor immune response, and contemplated ctRNAs thus include those encoding MUC1. In further examples, ctRNA that encode various cancer hallmark genes are contemplated. For example, where the hallmark is EMT (epithelial-mesenchymal transition), contemplated ctRNA may encode brachyury. In these and other cases (especially where secreted inhibitory factors are present), it is contemplated that upon detection of the ctRNA suitable therapeutic action may be taken (e.g., apheretic removal of such soluble factors, etc.). Further aspects and considerations for use in conjunctions with the teachings presented herein are described in WO 2016/077709, U.S. 62/513,706, filed 01-Jun.-17, U.S. 62/504,149, filed 10-May-17, and U.S. 62/500,497, filed 02-May-17, all of which are incorporated in their entirety by reference herein.

It should be appreciated that the results from cfRNA quantification can not only be used as an indicator for the presence or absence of a specific cell or population of cells that gave rise to the measured cfRNA, but can also serve as an additional indicator of the state (e.g., genetic, metabolic, related to cell division, necrosis, and/or apoptosis) of such cells or population of cells, and/or status of tumor microenvironment. Thus, the inventors further contemplate that the results from cfRNA quantification can be employed as input data in pathway analysis and/or machine learning models. For example, suitable models include those that predict pathway activity (or activity of components of a pathway) in a single or multiple pathways. Thus, quantified cfRNA may also be employed as input data into models and modeling systems in addition to or as replacement for RNA data from transcriptomic analysis (e.g., obtained via RNAseq or cDNA or RNA arrays).

In some embodiments, cfRNA quantification and/or identification of cfDNA/cfRNA mutation can be determined over time. Particularly where the cfRNA is quantified over time, it is generally preferred that more than one measurement of the same (and in some cases newly identified) mutation are performed. For example, multiple measurements over time may be useful in monitoring treatment effect that targets the specific mutation or neoepitope. Thus, such measurements can be performed before/during and/or after treatment. Where new mutations are detected, such new mutations will typically be located in a different gene and as such multiple and distinct cfRNAs are monitored.

Advantageously, contemplated methods are independent of a priori known mutations leading to or associated with a cancer. Still further, contemplated methods also allow for monitoring clonal tumor cell populations as well as for prediction of treatment success with an immunomodulatory therapy (e.g., checkpoint inhibitors or cytokines), and especially with neoepitope-based treatments (e.g., using DNA plasmid vaccines and/or viral or yeast expression systems that express neoepitopes or polytopes). In this regard, it should also be noted that the efficacy of immune therapy can be indirectly monitored using contemplated systems and methods. For example, where the patient was vaccinated with a DNA plasmid, recombinant yeast, or adenovirus, from which a neoepitope or polytope is expressed, ctRNA of such recombinant vectors may be detected and as such validate transcription from these recombinant vectors.

In addition, the inventors further contemplated that the increased expression of cfRNA along with a mutation (e.g., missense mutations, insertions, deletions, various fusions or translocations, etc.) in the cfDNA/cfRNA or the gene from which the cfDNA/cfRNA is derived from, may indicate that the cfDNA/cfRNA may be derived from a gene encoding a tumor antigen and/or patient- and tumor-specific neoepitope. Most typically, the patient-specific epitopes are unique to the patient, and may as such generate a unique and patient specific marker of a diseased cell or cell population (e.g., sub-clonal fraction of a tumor). Consequently, it should be especially appreciated that cfRNA carrying such patient and tumor specific mutation may be followed as a proxy marker not only for the presence of a tumor, but also for a cell of a specific tumor sub-clone (e.g., treatment resistant tumor). Moreover, where the mutation encodes a patient and tumor specific neoepitope that is used as a target in immune therapy, such the cfRNA carrying such mutation will be able to serve as a highly specific marker for the treatment efficacy of the immune therapy.

Consequently, the inventors further contemplate that a treatment regimen can be designed and/or determined based on the cancer status and/or the changes/types of cfDNA and/or cfRNA. It is contemplated that the likelihood of success of a treatment regimen may be determined based on the cancer status and the type/quantity of the cfDNA and/or cfRNA. For example, in some embodiments where the quantity of cfRNA derived from a gene expressed in the cell (e.g., tumor cell, immune cell, etc.) indicating immune suppressive tumor microenvironment, development of cancer sternness, onset of metastasis, or other cancer status, the protein or peptide encoded by the gene from which the cfRNA is derived can be targeted by an antagonist or any other type of binding molecule to inhibit the function of the peptide. Thus, increased expression (e.g., above a predetermined threshold) of cfRNA derived from the gene related to immune suppressive tumor microenvironment implicates the presence of immune suppressive tumor microenvironment, and also implicates that an antagonist to the peptide encoded by the gene related to immune suppressive tumor microenvironment has a high likelihood of success to inhibit the progress of the cancer by inhibiting immune suppressive tumor microenvironment and further promoting immune cell activity against tumor cells in such microenvironment. Any suitable antagonists to a target molecule are contemplated. For example, a specific kinase can be targeted by a kinase inhibitor, or a specific signaling receptor can be targeted by synthetic ligand, or a specific checkpoint receptor targeted by synthetic antagonist or antibody, etc. In other embodiments where the quantity of cfRNA derived from noncoding RNA increases, the treatment regimen may include any inhibitor(s) to the noncoding RNA (e.g., miRNA inhibitors such as another miRNA having a complementary sequence with the miRNA, etc.).

Further, where the cfDNA and/or cfRNA analysis indicates a presence of neoepitope expressed by tumor cells, a treatment regimen may include a neoepitope based immune therapy. Any suitable immune therapies targeting the neoepitope are contemplated, and the exemplary immune therapies may include an antibody-based immune therapy targeting the neoepitope with a binding molecule (e.g., antibody, a fragment of antibody, an scFv, etc.) to the neoepitope and a cell-based immune therapy (e.g., an immune competent cell having a receptor specific to the neoepitope, etc.). For example, the cell-based immune therapy may include a T cell, NK cell, and/or NKT cells expressing a chimeric antigen receptor specific to the neoepitope derived from the gene having the patient- and tumor-specific mutation.

The inventors further contemplated that the treatment regimen may include two or more pharmaceutical composition that targets two separate and/or distinct molecule related to the two or more cfRNA/cfDNA that show changes in the patient's sample. For example, patient's sample may have increased expression of one cfRNA derived from checkpoint inhibition related genes (e.g., PD-L1), and increased expression of another cfRNAs derived from CXCL1 and CXCL2 genes, respectively, that may indicate immune-suppressive tumor microenvironment by MDSC cell recruitment and deposition. In such example, the treatment regimen may include a checkpoint inhibitor and an antibody (or a binding molecule) against CXCL1 and/or CXCL2, which may be administered to the patient concurrently or substantially concurrently (e.g., same day, etc.), or which may be administered separately and/or sequentially (e.g., on different days, one treatment is administered after the series of administration of another treatment is completed, etc.).

Additionally, it is also contemplated that the cfDNAs and/or cfRNAs can be detected, quantified and/or analyzed over time (at different time points) to determine the effectiveness of a treatment to the patient and/or response of a patient or patient's tumor to the treatment (e.g., developing resistance, susceptibility, etc.). Generally, multiple measurements can be obtained over time from the same patient and same bodily fluid, and at least a first cfRNA may be quantified at a single time point or over time. Over at least one other time point, a second cfRNA may then be quantified, and the first and second quantities may then be correlated for monitoring treatment. In some embodiments, the first and second cfRNAs are same types of RNA and/or derived from the same gene to monitor changes of same type of cfRNA (e.g., PD-L1) upon treatment. In other embodiments, the first and second cfRNAs may be different types of RNA (e.g., one derived from mRNA and another derived from miRNA) and/or derived from the different genes. For example, the first ctRNA is derived from a tumor associated gene, a tumor specific gene, or covers a patient- and tumor specific mutation. Over at least one other time point, a second cfRNA may then be quantified, and the first and second quantities may then be correlated for diagnosis and/or monitoring treatment. In such example, the second cfRNA may also be derived from a gene that is relevant to the immune status of the patient, for example, a checkpoint inhibition related gene, a cytokine related gene, and/or a chemokine related gene, or the second cfRNA is a miRNA. Thus, contemplated systems and methods will not only allow for monitoring of a specific gene, but also for the status of an immune system. For example, where the second cfRNA is derived from a checkpoint receptor ligand or IL-8 gene, the immune system may be suppressed. On the other hand, where the second cfRNA is derived from an IL-12 or IL-15 gene, the immune system may be activated. Thus, measurement of a second cfRNA may further inform treatment. Likewise, the second cfRNA may also be derived from a second metastasis or a subclone, and may be used as a proxy marker for treatment efficacy. In this regard, it should also be noted that the efficacy of immune therapy can be indirectly monitored using contemplated systems and methods. For example, where the patient was vaccinated with a DNA plasmid, recombinant yeast, or adenovirus, from which a neoepitope or polytope is expressed, cfRNA of such recombinant vectors may be detected and as such validate transcription from these recombinant vectors.

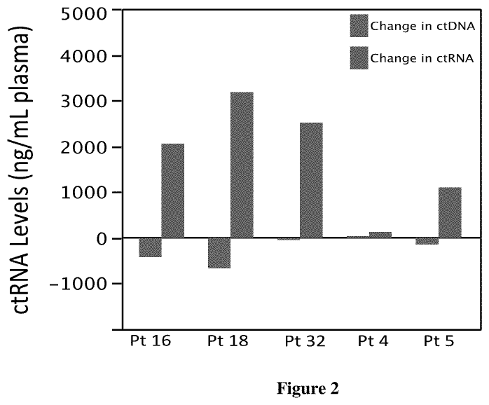

For example, as shown in FIG. 2, changes in total amount of cfRNA (or ctRNA) can be an indicative of emerging resistance to various therapies. Patient #16 was treated with a combination of Xeloda/Herceptin/Perjeta. Patient #18 was treated with a combination of Taxol/Carbo. Patient #32 was treated with a combination of Letrozole/Ibrance. Patient #4 was treated with Fulvestrant. Patient #5 was treated with a combination of Femara/Afinitor. Expression levels of total ctRNA from plasma of five patients progressing on various therapies were measured by RT-PCR, normalized by the expression level of beta-actin. Blood draws were taken approximately six weeks apart. While the changes in ctDNA levels in the patients' serum in 6 weeks after the treatment were not significantly changed, total ctRNA levels in patient #16, #18, #32, and #5 were significantly increased, indicating that the treatment(s) administered to those patients were effective to attack the cancer cell or increase immune response against the cancer cells. Meanwhile, it is shown that in patient #4, neither ctDNA level nor ctRNA level were changed significantly after treatment, suggesting that Fulvestrant administration to patient #4 was not effective or cancer cells of patient #4 developed resistance to Fulvestrant treatment.

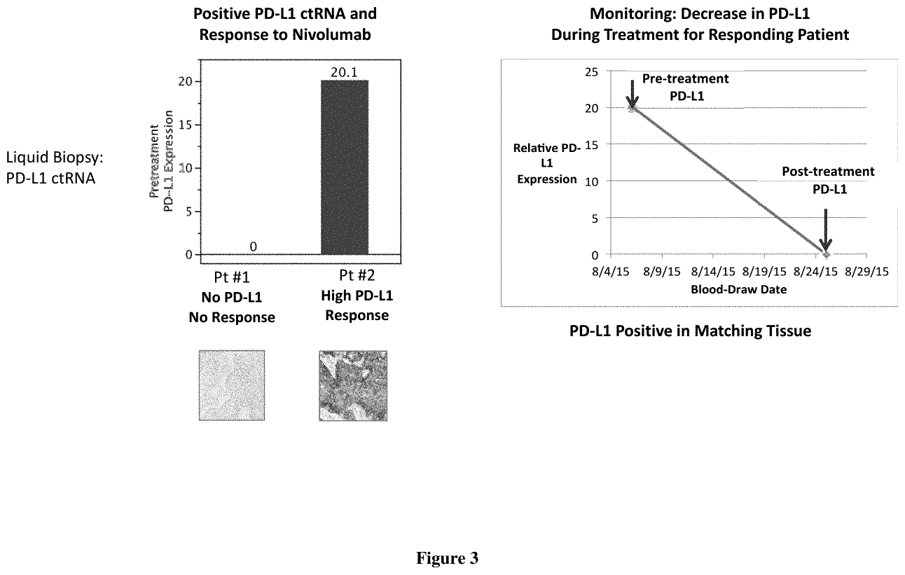

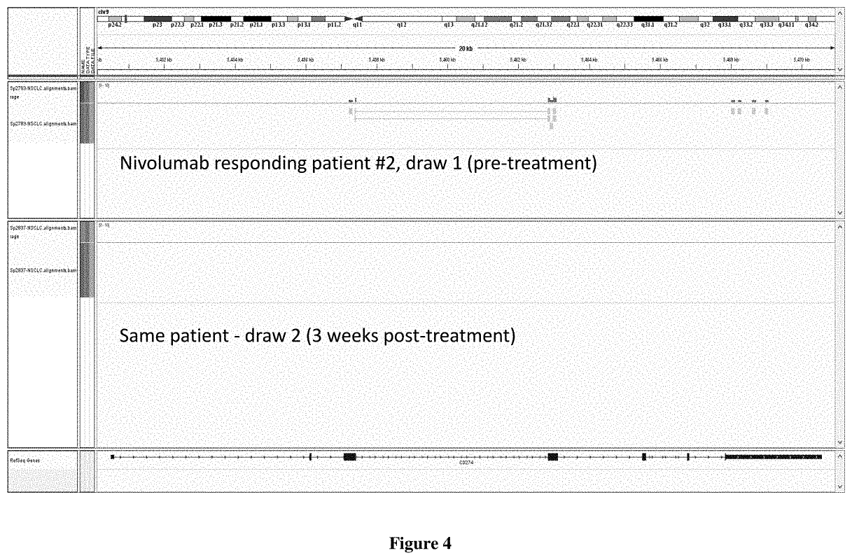

In another example, the difference in PD-L1 status (i.e., PD-L1 positive or PD-L1 negative) of two selected patients (Pt #1 and Pt #2) also correlated well with IHC analysis and treatment response with nivolumab as can be seen from FIG. 3. Here, two squamous cell lung cancer patients were treated with the anti-PD-1 antibody nivolumab. Patient 1 had no expression of PD-L1 in the tissue or in the blood using cfRNA measurement, suggesting that Patient 1 did not respond to nivolumab. Tumor growth was documented by CT scan and the patient expired rapidly. In contrast, Patient 2 had high levels of PD-L1 in the tissue and in the blood at baseline using cfRNA measurement. Patient 2 responded to nivolumab with a durable response over several cycles of the drug. The response was documented by CT scan with dramatic tumor shrinkage. Interestingly, the high levels of gene expression in the blood of this patient (measured by cfRNA) disappeared after three and a half weeks while the patient continued to respond. Such tumor shrinkage is consistent with RNA-seq and QPCR results obtained from patient #2 as shown in FIG. 4. In Nivolumab-responding patient #2, in the pre-treatment, PD-L1 ctRNA expression was positive shown as sequence aligned with the gene at or near q11 and q21.32. In the second blood drawing (3 weeks post treatment) from the same patient (patient #2), PD-L1 ctRNA expression level is almost undetectable (negative), consistent with the dramatic tumor shrinkage supplementarily evidenced by CT scan.

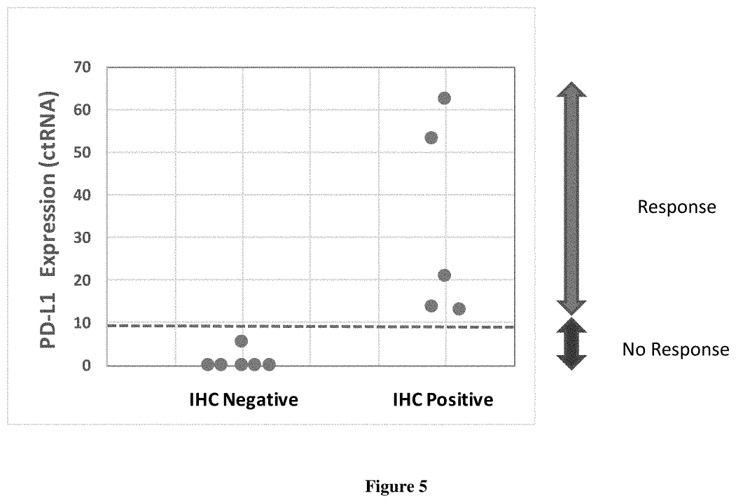

Based on the above observed correlation, the inventors set out to investigate whether or not expression levels of PD-L1 cfRNA could provide threshold levels suitable for response prediction to treatment with nivolumab or other therapeutics interfering with PD1/PD-L1 signaling. To that end, PD-L1 expression was measured in NSCLC patient plasma using cfRNA and compared with IHC status. FIG. 5 shows the correlation between treatment response status with an anti-PD-L1 therapeutic and PD-L1 status as determined by IHC and PD-L1 expression above response threshold by cfRNA. Patients determined to be treatment responders were also determined by IHC as PD-L1 positive, while all patients determined to be non-responders to treatment were determined by IHC as PD-L1 negative. Remarkably, the same separation between responders and non-responders could be achieved using PD-L1 cfRNA levels when a response threshold was applied to then data. In this example, a relative expression threshold of 10 accurately separated responders from non-responders.

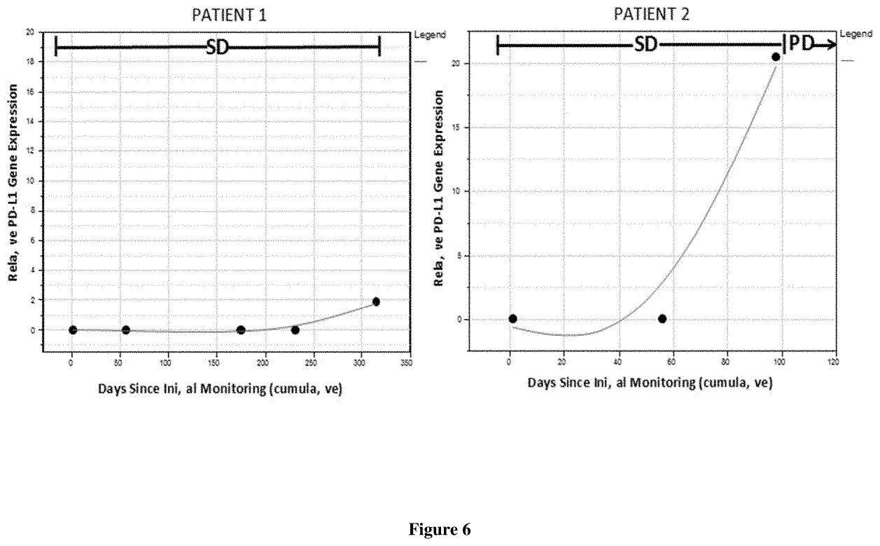

Further, the inventors measured expression levels of PD-L1 cfRNA to determine the progress or status of the cancer. As shown in FIG. 6, expression levels of PD-L1 cfRNA Patient #1 and #2 treated with Nivolumab were monitored about 350 days in patient #1, and about 120 days in patient #2. Stable levels of relative PD-L1 expression corresponded with stable disease status (SD). Subsequent rises in PD-L1 levels were predictive of resistance to Nivolumab therapy, which could be detectable by CT scans at least 1.5 months later.

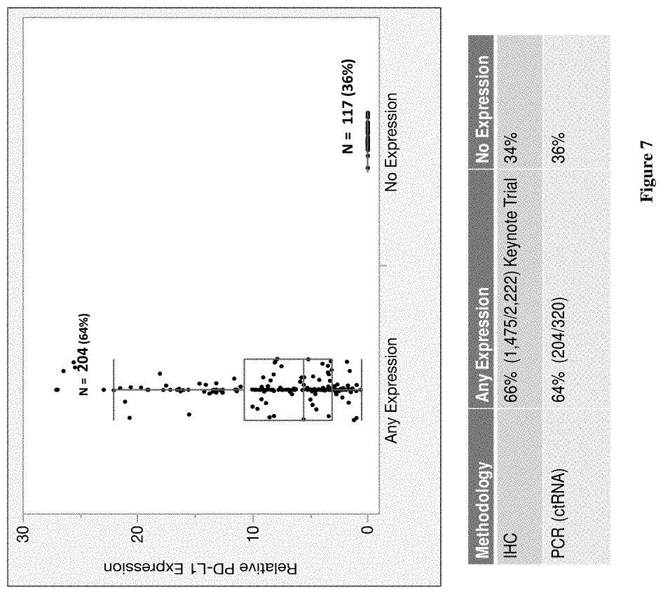

Based on the above findings that cfRNA can be accurately quantified, the inventors sought to determine whether the quantified cfRNA levels would also correlate with known analyte levels measured by conventional methods such as FISH, mass spectroscopy, etc. More specifically, the frequency and strength of PD-L1 expression was measured by cfRNA from the plasma of 320 consecutive NSCLC patients using LiquidGenomicsDx and compared to the frequency of positive patients in the Keynote Trial, a registration trial of pembrolizumab (Keytruda), using a tissue IHC test. Notably, 66% of NSCLC patients (1,475/2,222) in the Keynote trial had any expression of PD-L1 by IHC (>1% of cells positive), while 64% of NSCLC (204/320) patients with blood-based cfRNA testing of PD-L1 were positive as can be seen from FIG. 7. Remarkably, there was no significant difference in PD-L1 status between the two analytical methods, but the cfRNA testing afforded quantitative data.

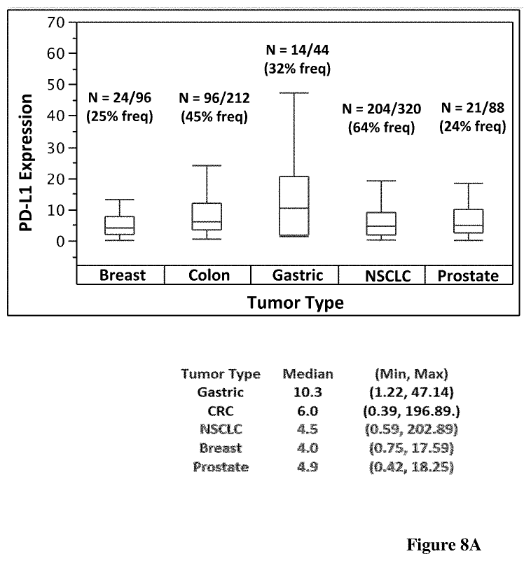

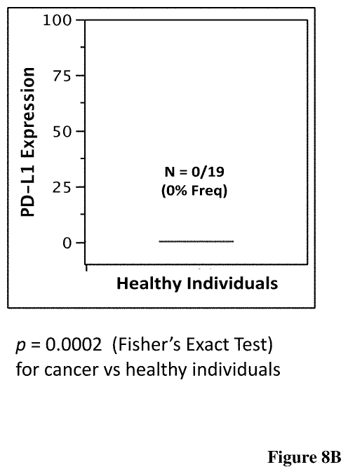

The inventors further investigated whether the above results could be confirmed across various other cancer types and selected genes (e.g., PD-L1) and analyzed blood samples from selected patients diagnosed with breast cancer, colon cancer, gastric cancer, lung cancer, and prostate cancer. In this series of tests, relative expression of PD-L1cfRNA was quantitated, and the results are depicted in FIG. 8A. Interestingly, not all cancers expressed PD-L1 as shown in FIG. 2A, and the frequencies of positivity in the various cancers was concordant with the published expression of PD-L1 using IHC in solid tissue. PD-L1cfRNA was not detectable in healthy patients as can be seen from FIG. 8B.

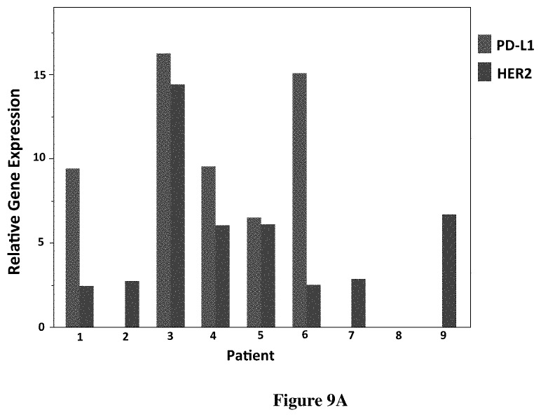

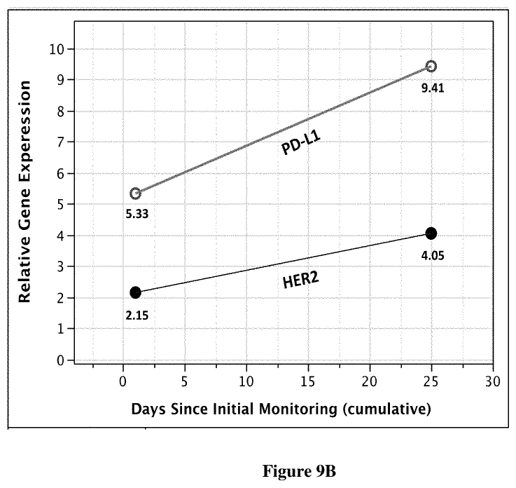

Upon further investigation of breast cancer samples, the inventors also discovered that HER2 cfRNA in tumors appeared to be co-expressed or co-regulated with PD-L1 as is shown in FIG. 9B. Additionally, the inventors also discovered that that HER2 cfRNA in at least some gastric tumors also appeared to be co-expressed or co-regulated with PD-L1 as is shown in FIG. 9A. Such finding is particularly notable as it is known that about 15% of all gastric cancers do express HER2. Consequently, the inventors contemplate methods of detecting or quantifying HER2 cfRNA in patients with gastric cancer. Furthermore, the inventors also contemplate that one or more immune checkpoint genes (e.g., PD-L1, TIM3, LAG3) as measured by cfRNA may be used as proxy markers for other cancer specific markers or tumor associated markers (e.g., CEA, PSA, MUC1, brachyury, etc.).

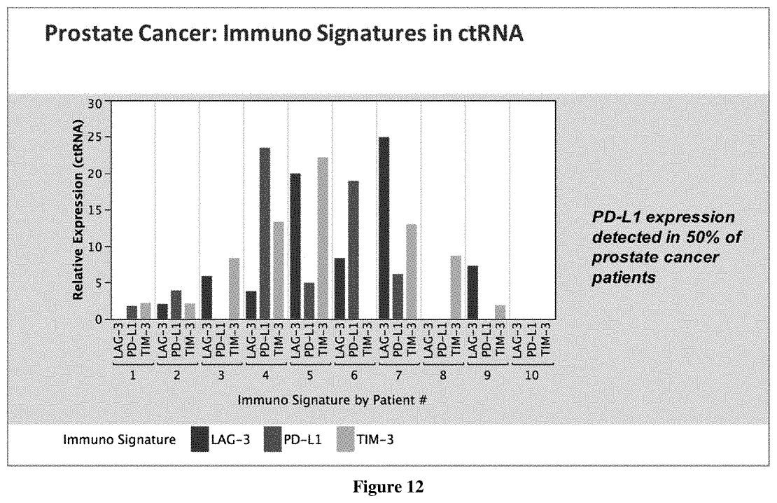

Based on the observed co-expression or co-regulation, the inventors then investigated whether or not other cfRNA levels for immune checkpoint related genes would correlate with PD-L1 cfRNA levels and exemplary results are depicted in FIG. 12. Here, cfRNA levels for PD-L, TIM3, and LAG3 were measured from blood samples of prostate cancer patients. Notably, in all but one sample more than one checkpoint related gene was strongly expressed. Interestingly and importantly, levels of TIM3 and LAG3, the former of which has been shown to serve as an escape mechanism or resistance factor for PD-1 or PD-L1 inhibition, often mirrored PD-L1 expression, underscoring a need to address all checkpoint proteins besides PD-1 and PD-L1. Therefore, it should be appreciated that cfRNA levels for immune checkpoint relevant genes may be analyzed for cancer patients to so obtain an immune signature or the patient, and the appropriate treatment with more than one checkpoint inhibition drug may be then be advised. As will be appreciated, suitable threshold values for the genes can be established following the methods described for PD-L1 and HER2 above.

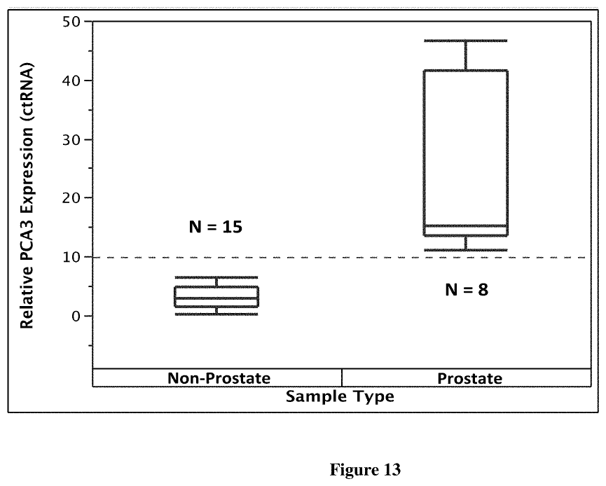

Furthermore, PCA3 was identified as a marker for prostate cancer in a test in which PCA3 cfRNA was detected and quantified in plasma from prostate cancer patients and in which non-prostate cancer patient samples had relatively low to non-detectable levels. Non-prostate cancer patients were NSCLC and CRC patients. As can be taken from FIG. 13, PCA3 was shown to be differentially expressed between the two groups (non-overlapping medians between prostate and non-prostate cancer patients) by cfRNA, indicating that the non-invasive blood based cfRNA test may be used to detect prostate cancer. Once more, based on a priori knowledge of the tested population, a threshold value (here: ΔΔCT>10 for PCA3 relative to β-actin) for expression could be established as is exemplarily depicted in FIG. 13.

Alternatively and/or additionally, it is also contemplated that the each of first and second cfRNAs are sets of cfRNAs that may comprise a plurality of cfRNAs derived from a plurality of genes, respectively, among which some of them may be common. For example, the first cfRNA may include cfRNAs derived from genes A, B and C, respectively, and the second cfRNA may include cfRNAs derived from genes A, D, and E, respectively. In another example, the first cfRNA may include cfRNAs derived from genes A, B and C, respectively, and the second cfRNA may include cfRNAs derived from genes D, E, and F, respectively. Thus, the first set of cfRNAs may be associated with immune suppressive tumor microenvironment, and the second set of cfRNAs may be associated with metastasis/EMT.

Thus, it should be appreciated that cfRNA of a patient can be identified, quantified, or otherwise characterized in any appropriate manner. For example, it is contemplated that systems and methods related to blood-based RNA expression testing (cfRNA) that identify, quantify expression, and allow for non-invasive monitoring of changes in drivers of disease (e.g., PD-L1 and nivolumab or pembrolizumab) be used, alone or in combination with analysis of biopsied tissues. Such cfRNA centric systems and methods allow monitoring changes in drivers of a disease and/or to identify changes in drug targets that may be associated with emerging resistance to chemotherapies. For example, cfRNA presence and/or quantity of one or more specific gene (e.g., mutated or wild-type, from tumor tissue and/or T-lymphocytes) may be used as a diagnostic tool to assess whether or not a patient may be sensitive to one or more checkpoint inhibitors, such as may be provided by analysis of cfRNA for ICOS signaling.

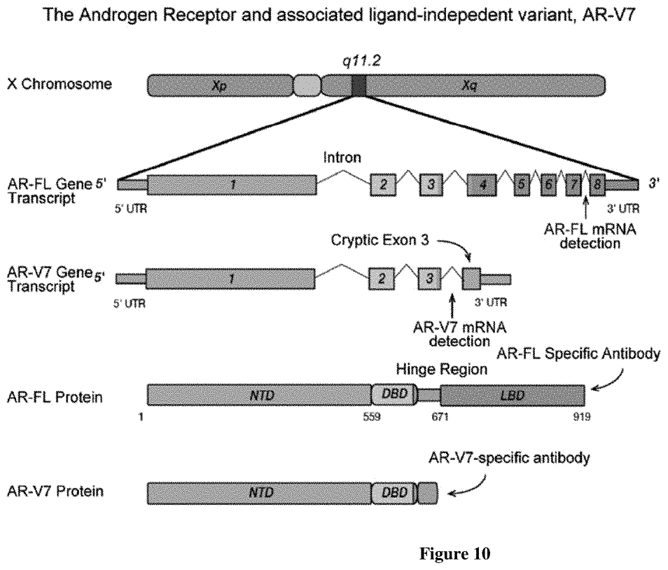

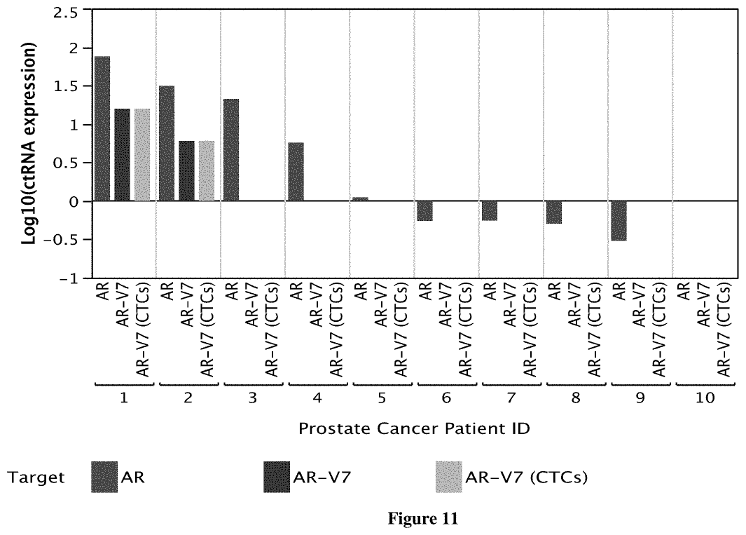

Furthermore, various alternate cfRNA species can be detected to quantitatively distinguish healthy individuals from those afflicted with cancer and/or to predict treatment response. As shown in FIG. 10, androgen receptor gene can be transcribed into multiple splicing variants, one of which is translated into splice variant 7 of the androgen receptor (AR-V7) protein. The detection of the splice variant 7 of the androgen receptor (AR-V7) has been an important consideration for the treatment of prostate cancer with hormone therapy. The inventors therefore investigated whether or not hormone therapy resistance is associated with prostate cancer tumor growth and detection of AR-V7 via detection and quantification of AR-V7 cfRNA. FIG. 11 depicts exemplary results for AR and AR-V7 gene expression via cfRNA methods using plasma from prostate cancer patients. AR-V7 was also measured using IHC technology from circulating tumor cells (CTCs from the same patients. Notably, the results from CTCs and cfRNA for AR-V7 were concordant.

Moreover, and viewed from yet another perspective, the inventors also contemplate that contemplated systems and methods may be employed to generate a mutational signature of a tumor in a patient. In such method, one or more cfRNAs are quantified where at least one of the genes leading to those cfRNAs comprises a patient- and tumor-specific mutation. Such signature may be particularly useful in comparison with a mutational signature of a solid tumor, especially where both signatures are normalized against healthy tissue of the same patient. Differences in signatures may be indicative of treatment options and/or likelihood of success of the treatment options. Moreover, such signatures may also be monitored over time to identify subpopulations of cells that appear to be resistant or less responsive to treatment. Such mutational signatures may also be useful in identifying tumor specific expression of one or more proteins, and especially membrane bound or secreted proteins, that may serve as a signaling and/or feedback signal in AND/NAND gated therapeutic compositions. Such compositions are described in copending US application with the Ser. No. 15/897,816, which is incorporated by reference herein.

Among various other advantages, it should be appreciated that use of contemplated systems and methods simplifies treatment monitoring and even long term follow-up of a patient as target sequences are already pre-identified and target cfRNA can be readily surveyed using simple blood tests without the need for a biopsy. Such is particularly advantageous where micro-metastases are present or where the tumor or metastasis is at a location that precludes biopsy. Further, it should be also appreciated that contemplated compositions and methods are independent of a priori knowledge on known mutations leading to or associated with a cancer. Still further, contemplated methods also allow for monitoring clonal tumor cell populations as well as for prediction of treatment success with an immunomodulatory therapy (e.g., checkpoint inhibitors or cytokines), and especially with neoepitope-based treatments (e.g., using DNA plasmid vaccines and/or viral or yeast expression systems that express neoepitopes or polytopes).

With respect to preventative and/or prophylactic use, it is contemplated that identification and/or quantification of known cfDNAs and/or cfRNAs may be employed to assess the presence or risk of onset of cancer (or other disease or presence of a pathogen). Depending on the particular cfRNA detected, it is also contemplated that the cfDNAs and/or cfRNAs may provide guidance as to likely treatment outcome with a specific drug or regimen (e.g., surgery, chemotherapy, radiation therapy, immunotherapeutic therapy, dietary treatment, behavior modification, etc.). Similarly, quantitative cfRNA results may be used to gauge tumor health, to modify immunotherapeutic treatment of cancer in patient (e.g., to quantify sequences and change target of treatment accordingly), or to assess treatment efficacy. The patient may also be placed on a post-treatment diagnostic test schedule to monitor the patient for a relapse or change in disease and/or immune status.

Thus, the inventors further contemplate that, based on cfDNAs and/or cfRNAs detected, analyzed, and/or quantified, a new treatment plan can be generated and recommended or a previously used treatment plan can be updated. For example, a treatment recommendation to use immunotherapy to target a neoepitope encoded by gene A can be provided based on the detection of ctDNA and/or ctRNA (derived from gene A) and increased expression level of ctRNA having patient- and tumor-specific mutation in gene A, which is obtained from the patient's first blood sample. After 1 month of treatment with an antibody targeting the neoepitope encoded by gene A, the second blood sample was drawn, and ctRNA levels were determined. In the second blood sample, ctRNA expression level of gene A is decreased while ctRNA expression level of gene B is increased. Based on such updated result, a treatment recommendation can be updated to target neoepitope encoded by gene B. Also, the patient record can be updated that the treatment targeting the neoepitope encoded by gene A was effective to reduce the number of tumor cells expressing neoepitope encoded by gene A.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of determining a cancer status in an individual having or suspected to have a cancer, comprising:
obtaining a sample of a bodily fluid of the individual;
determining a quantity of a cfRNA in the sample, wherein the cfRNA is derived from a cancer related gene; and
associating the quantity of the cfRNA with the cancer status, wherein the cancer status is at least one of presence of metastasis, presence of cancer stem cells, presence of immune suppressive tumor microenvironment, and increased or decreased activity of an immune competent cell against the cancer.

2-46. (canceled)

47. A method of treating a cancer, comprising:
determining quantities of at least one of respective cfRNA and ctRNA of first and second marker genes in a blood sample of a patient;
wherein the first marker gene is a cancer related gene, and wherein the second marker gene is a checkpoint inhibition related gene;
using the quantity of the cfRNA or ctRNA derived from the first marker gene to determine treatment with a first pharmaceutical composition;
using the quantity of the cfRNA or ctRNA derived from the second marker gene to determine treatment with a second pharmaceutical composition; and
wherein the second pharmaceutical composition comprises a checkpoint inhibitor.

48. The method of claim 47, wherein the second marker gene encodes PD-1 or PD-L1.

49-61. (canceled)

62. The method of claim 47, further comprising determining a total quantity of all cfRNA and ctRNA in the sample, and optionally using the determined total quantity to determine treatment with a third pharmaceutical composition.

63. The method of claim 47, further comprising determining at least one of presence and quantity of a soluble NKG2D ligand in the bodily fluid.

64. The method of claim 47, wherein the step of determining includes isolation of the at least one of cfRNA and ctRNA under conditions and using RNA stabilization agents that substantially avoids cell lysis.

65. The method of claim 47, wherein the cancer related gene is a cancer associated gene, a cancer specific gene, a cancer driver gene, or a gene encoding a patient and tumor specific neoepitope.

66. The method of claim 65, wherein the cancer related gene is selected form the group consisting of ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCB5, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGR5, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, MICA, MICB, MBLL, ULBP1, ULBP2, ULBP3, ULBP4, ULBP5, ULBP6, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, XCR1, DCC, UNC5A, Netrin, and IL8.

67. The method of claim 66, wherein the cancer related gene has a patient-specific mutation or a patient- and tumor-specific mutation, and wherein the mutation is at least one of a missense mutation, an insertion, a deletion, a translocation, and a fusion.

68. The method of claim 67, wherein the at least one of the ctRNA and cfRNA is a portion of the cancer related gene encoding a patient-specific and cancer-specific neoepitope.

69. The method of claim 47, wherein the treatment with the first pharmaceutical composition is based on a first cancer status determined by the quantity of the cfRNA or ctRNA derived from the first marker.

70. The method of claim 69, wherein the first cancer status is at least one of the following: susceptibility of the cancer to treatment with a drug, presence or absence of the cancer in the individual, presence of metastasis, presence of cancer stem cells, presence of immune suppressive tumor microenvironment, and increased or decreased activity of an immune competent cell against the cancer.

71. The method of claim 47, further comprising determining quantities of at least one of respective cfRNA and ctRNA derived from first and second marker genes in a plurality of blood samples of a patient obtained after treating the patients with at least one of the first and second pharmaceutical compositions.

72. The method of claim 71, further comprising determining effectiveness of the at least one of the first and second pharmaceutical compositions based on at least one of the quantities of at least one of respective cfRNA and ctRNA.

73. The method of claim 72, further comprising modifying a treatment plan to replace at least one of the first and second pharmaceutical compositions with a fourth pharmaceutical composition.

74. The method of claim 47, wherein the at least one of cfRNA and ctRNA is a miRNA to the first second marker gene, and the first pharmaceutical composition is an inhibitor to the miRNA.

75-120. (canceled)

121. A method of determining a likelihood of success of an immune therapy to an individual having a cancer, comprising:
obtaining a sample of a bodily fluid of the individual;
determining a quantity of at least one of cfRNA and ctRNA in the sample, wherein the cfRNA and ctRNA is derived from at least one of an epithelial to mesenchymal transition-related gene and an immune suppression-related gene;
associating the quantity of the at least one of cfRNA and ctRNA with a tumor microenvironment status; and
determining the likelihood of success of the immune therapy based on a type of the immune therapy and the tumor microenvironment status.

122-150. (canceled)
